(19) United States
(12) Patent Application Publication
(10) Pub. No.: US 2026/0232414 A1
(43) Pub. Date: Aug. 13, 2026

(54) METHOD, SYSTEM AND COMPOUND FOR THE AUTOMATED, SOFTWARE-BASED MANUFACTURING OF A VENEER OF A FIXED DENTAL PROSTHESIS

(71) Applicant: Gernot TEICHMANN, Luzern (CH)

(72) Inventor: Gernot TEICHMANN, Luzern (CH)

(21) Appl. No.: 19/052,026

(22) Filed: Feb. 12, 2025

(51) Int. Cl. A61C 13/00; A61C 5/73; A61C 5/77

(52) U.S. Cl. CPC A61C 13/0019; A61C 5/73; A61C 5/77; A61C 13/0004

(57) ABSTRACT

A method and a system for the automated, software-based manufacturing of a veneer of a fixed dental prosthesis by coating a dental preform with at least one compound forming a ceramic paste that reproduces the dentin of a natural tooth. The preform is coated based on a 3D layer structure in which each layer is divided into volume elements. Each volume element is filled with a portion of the ceramic paste by aligning the preform with a manipulator so that a ground surface of the volume element to be filled, at an application point on the ground surface, is oriented substantially perpendicular to an ejection direction with the application point lying on the ejection direction. A print head applies the respective portion of the paste by jerking a valve element so that the portion is formed and projected in a coherent manner toward the application point and fills the volume element in a punctiform manner. The compound used is a ceramic paste formed from a powder of a feldspar ceramic finely dispersed in a solvent and including amounts of ammonium polyacrylate, ethoxylated castor oil and traces of urea.

### TECHNICAL FIELD OF THE INVENTION

The invention relates to the technical field of manufacturing a dental prosthesis. In particular, the invention relates to a method, a system and at least one compound for the automated, software-based manufacturing of a veneer of a fixed dental prosthesis by coating a dental preform with the at least one compound which serves to reproduce the dentin of a natural tooth, wherein the compound is a ceramic paste which is formed from a powder of a feldspar ceramic finely dispersed in a solvent. The invention also relates to the use of the compound in the method and the system.

### STATE OF THE ART

A natural tooth 1, a cross-sectional view of which is shown in FIG. 1, can be divided spatially into a tooth head 1a and a tooth root 1b. The tooth head 1a is the visible part of the tooth 1. It protrudes from the gum and projects into the oral cavity, while the tooth root 1b extends into the jawbone. The root 1b contains the nerve 4 of the tooth inside. Tooth head 1a and tooth root 1b consist of dentin 3, which is covered in the area of the tooth head 1a with dental enamel 2, also known as the enamel layer. The enamel 2 therefore forms the natural outer layer of tooth 1, while the dentin 3 is on the inside. Bacteria on tooth 1 can damage the enamel 2 and the underlying dentin 3 by decomposition of food debris and the sugars it contains on the tooth surface, creating an acid that attacks the enamel 2 and gradually erodes the tooth 1 if it is not cleaned regularly. This disease of a tooth is known as “caries”. Caries initially manifests itself as whitish or brownish spots on the teeth. As the disease progresses, holes appear on the surface of the teeth. If the deeper areas of the tooth 1 are affected, caries also damages the tooth nerves 4 and the tooth roots 1b. To treat tooth decay, the areas of the tooth 1 affected by the acid must be removed.

While caries-related holes in dentin 3 can be easily drilled out and filled with fillings made of plastic (composite), amalgam, ceramic or precious metal, tooth 1 can also be so severely damaged that a filling is not possible. In this case, the tooth is ground down in such a way that only a tooth trunk 1c, i.e. trunk-shaped or semi-trunk-shaped healthy inner area of the dentin 3 remains, on which a dental prosthesis 5, 5a in the form of a crown 5, 5a or partial crown replacing the natural tooth head 1a can then be placed. FIGS. 2 and 3 each show such a case of a crowned tooth 1′. Here, only a tooth trunk 1c of the dentin 3 protrudes into the oral cavity from the tooth root 1b located in the jawbone. A crown 5, which in FIG. 2 consists of a preform 6 with a hard ceramic base, for example zirconium dioxide, a veneer 9 made of a dental ceramic on the preform 6 and an outer gloss layer 10 on the dental ceramic 9, and the crown 5 is bonded to the tooth stump 1c.

FIG. 3 shows another crown 5a, which also consists of a preform 6, a veneer 9 made of a dental ceramic 9 applied to the preform 6 and an outer gloss layer 10, but here the preform 6 is made from a metallic base 6a. This metallic base 6a is first coated with a bonding agent 7, a so called bonder, to improve the adhesion of the veneer 9. The bonding agent 7 forms a primer that is sprayed on the preform 6 as an aerosol using an air brush. The bonding agent 7 has a layer thickness of between 20 μm and 50 μm and is also fired in an oven. A color layer 8, known in the circles of experts as an “opaquer”, is applied to the bonding agent 7 in order to cover the color of the metal of the preform, as the metallic base 6a appears black-grey through the translucent bonding agent 7. The color layer 8 is intended to have the same shade as the dentin 3. The color layer 8 does not have to be fired in an oven on its own. It is sufficient if it is fired together with the dental ceramic forming the veneer 9 after firing.

In an incisor, the dentin 3 of the natural tooth 1 becomes more transparent towards the cutting edge. The layer of dental enamel 2 covering the dentin 3 is considerably thicker than the gloss layer 10 that is applied to artificial crowns 5, 5a at the end of the manufacturing process. This enamel layer 2 is very transparent.

The gloss layer 10 of the crown 5, 5a optically fulfills the same function as the dental enamel 2 of the natural tooth 1. The gloss layer 10 is sprayed on as an aerosol after coating the preform 6 with dental ceramic 9. It is only a few fractions of a millimeter thick, for example approx. 100 μm.

The natural Tooth 1 may also be so severely damaged that it has to be removed. In this case, it can be replaced by a jaw implant with a crown placed on it, whereby the implant forms an artificial tooth root and the crown 5, 5a, as can be seen in FIGS. 2 and 3, replaces the exposed part of natural tooth 1 protruding from the gum. Alternatively, the natural tooth 1 can be replaced by a dental prosthesis in the form of a pontic 5b (bridge), in which the tooth 1 to be reproduced forms a central region, while the end regions of the pontic 5b are attached to the neighboring natural teeth of the replaced natural tooth 1. Crowns 5, 5a and pontics 5b form a fixed dental prosthesis, i.e. they are to be distinguished from removable dental prostheses.

The enamel layer of a tooth is very hard and looks almost glass-like transparent. The dentin, on the other hand, gives the tooth its natural color. It is only slightly light-transmissive, but not opaque. The degree of light-transmissive capability is referred to as translucence. In addition, there are local optical effects in dentin that are caused by special crystal structures. These optical effects are called opalescence.

Due to the fact that every dental prosthesis is unique due to its outer shape, size and coloring, the production of the visible part of dental prostheses is still almost exclusively carried out manually by dental technicians, who crafts and coats, or more precisely paints, the preform with the dental ceramic manually by means of a brush, which act is known as “veneering”. Accordingly, the dental ceramic, after being applied on the preform directly or, due to intermediate layers 7, 8 indirectly, forms a veneer. After veneering, the dental ceramic is fired, more specifically sintered, in an oven (furnace) at more than 800° C.

The aim in crafting a dental prosthesis is to imitate the optical properties of the tooth or teeth to be replaced, namely the color, translucency and opalescence, in such a way that the dental prosthesis cannot be visually distinguished from the natural tooth or teeth. This goal is often not achieved using a dental ceramic made of a ceramic, such as zirconium oxide (ZrO₂) which is due to the lack of shade accuracy of the oxide ceramic. In addition, translucency and opalescence are completely lacking and the dental prosthesis appears bright white-comparable to a sanitary ceramic so that the artificial tooth or teeth look artificial and dead and is therefore esthetically unsatisfactory.

The translucency of the dental ceramic after firing is mainly achieved through the use of quartz in the ceramic powder mixture. The dental ceramic is therefore basically a glass ceramic. The addition of oxides of the alkaline group and/or the earth alkaline group can improve the translucency. The firing temperature also plays a role, which must not be too high or too low, generally between 850° C. and 950° C. Moreover, opalescence can be influenced by the incorporation of leucite crystals (KAlSiO₂) , which grow during firing. Traces of phosphorus can also increase the opalescence. Coloring of the dental ceramic is achieved by adding very small amounts of ceramic colorants based on zirconium, rutile or spinel. Minimum requirements for dental ceramics are defined in the EN ISO 6872(2019 -01) standard, which relates to ceramic materials in dentistry.

Coating the preform with the dental ceramic, i.e. the act of veneering, is also complicated by the fact that the dental ceramic must be specially prepared for this purpose by mixing a ceramic powder with a solvent, or more precisely a liquid, usually water, to form a slurry. Note that, in the following, the dental ceramic is also called slurry. If too much liquid is used for this, the slurry is so liquid in its processing state that it runs downwards at the preform due to gravity. This makes it impossible to produce elevated structures. If, on the other hand, too little liquid is used, the dental ceramic must be processed as quickly as possible as it dries in the air, and if it is then too dry, it cannot be shaped well and a new dental ceramic layer will not bond with an already applied dental ceramic layer. A very simplified analogy of this problem is that which refers to the optimum viscosity of a toothpaste: If the viscosity is too high, it is difficult to squeeze the paste out of the tube; if it is too low, the paste runs off the toothbrush. In practice, it is challenging to mix the ceramic powder and the liquid in such a ratio that the dental ceramic is in an optimal pasty state for processing, i.e. in such a way that it is viscous enough not to flow away but still malleable (plastically shapeable). Veneering is therefore a time-consuming and cumbersome activity. From an economic point of view, it is a very cost-intensive processing step, which is why manufacturing a dental prosthesis is comparatively expensive.

In the state of the art, various measures are used in an attempt to circumvent the conflicting requirements for the viscosity of pasty dental ceramics (sufficiently fluid for plastic shaping vs. not too fluid to prevent run-off). In European patent application EP 2233449 A1, for example, it is proposed to add a wax that is solid at room temperature to a highly diluted paste, whereby the mixture of paste and wax is heated and applied to the preform by spraying in the heated, low-viscosity state. The applied paste and wax mixture then cools down on the preform quickly, preventing it from flowing away. In the European patent applications EP 3919092 A1 and EP 3147707 A1, a light-curing monomer is added to the paste which, after application to the preform, causes the low-viscosity paste to harden by means of high-energy light beams of a specific wavelength. However, both methods have the disadvantage that the additives added, i.e. the wax on the one hand and the monomer on the other, have an extremely negative effect on the optical properties of the dental ceramic during firing. They are therefore unusable for esthetically sophisticated, high quality dental ceramic coatings.

The process proposed in the German patent application DE 102006015014 A1 avoids the viscosity problem by using a drop-on-demand inkjet printer in which a ceramic-containing ink has a viscosity of less than 55 mPa×s. The ink is a suspension of a dispersing medium and ceramic particles suspended therein, wherein the dispersing medium contains an aqueous boehmite sol (aluminum hydrate) as an essential component. However, the use of boehmite sol as a dispersing medium precludes its use for aesthetic purposes, as it leads to opacity and color shifts. The actual ceramic base material here is zirconium oxide with its well-known esthetic disadvantages. In addition, the system described can only print on a flat x-y plane; it is not possible to coat complex shapes.

A similar process is described in the study “Direct Inkjet Printing of Dental Prostheses Made of Zirconia” by J. Ebert et al., published in the Journal of Dental Research, August 2009, 88(7):673-6. This concerns the fabrication of a crown using a modified thermo-activated drop-on-demand desktop printer by depositing microscopic droplets of a zirconium oxide-based suspension. However, the rheological properties of zirconium oxide based suspensions are much better for ejection from microscopic nozzle openings than those of a feldspar-based suspension. The zirconium oxide crown produced did not have to meet any esthetic criteria. Nor did this study involve a coating.

A further problem of the normally used dental ceramics compared to automated procedures cited as prior art is that the shape of the particles (grains) forming the ceramic powder is, from a microscopic point of view, jagged and splintey. In addition, the individual particles vary greatly in size, ranging from 5 μm to 100 μm. For these reasons, the pasty dental ceramics in the prior art (handcrafted) are not suitable for every processing method, in particular they cannot be extruded or sprayed in small quantities.

The German patent application DE 102009011175 A1 describes a method for the automatic dental ceramic veneering of preforms of dental prosthesis, in particular for the veneering of crowns or pontics, in which it is provided that in at least one coating process at least one ceramic veneering material is sprayed onto the preform by a coating unit, the preform being held and moved by a holding and positioning unit during the coating process. Spraying requires the use of a low-viscosity material for veneering a preform, whereby DE 102009011175 A1 considers pressure atomization, ultrasonic atomization or even an electrospraying process to be suitable for application. In order words the application of the dental ceramic is effected by atomization so that finest droplets of the low-viscosity ceramic veneering material are produced and deposited on the preform. Due to the required low viscosity, the veneering material has a high liquid content. But unfortunately, spraying of a slurry with granules of the needed size is not possible because of the microstructure of the granules. It would be possible only with rounded granules with a diameter smaller than 2 μ. And a slurry containing said granules is not sticky enough; it runs down like water and is therefore not suited for that issue.

The German patent application DE 102009011175 A1 also describes pre-drying of an applied ceramic material on a dental object by means of a hot air supply.

European patent application EP 1252867 A1 constitutes a further prior art. It describes a method for producing an artificial tooth which has a base layer which substantially determines strength and corresponds to the aforementioned preform, and at least one cover layer which essentially determines the appearance of a finished artificial tooth and corresponds to the aforementioned dental ceramic, i.e. the veneer. The method comprises:
establishing the available outer shape and dimensions for the finished artificial tooth, e.g with a CAD-CAM system;
acquiring an image of a natural tooth to be replaced by the artificial tooth or a corresponding natural tooth over at least the in use visible outer surface thereof, including variations in appearance in said outer surface;
determining variations in the appearance determining properties of the cover layer to correspond with the variations in the appearance of the natural tooth to be replaced or the corresponding natural tooth, and determining a thickness of the cover layer locally required for said correspondence;
constructing the base layer to a shape and dimensions, which are based on the shape and dimensions available for the artificial tooth and prior to construction deducting, in the CAD-CAM system therefrom the locally required thickness of the cover layer; and
applying the cover layer over the base layer to attain the desired outer shape and dimensions for the finished artificial tooth.

In the method described here, the volume to be applied and the shape of the dental ceramic coating are calculated from the file for the final shape of the dental prosthesis created using a CAD-CAM system and a file for the preform of the dental prosthesis, by means of subtraction. The base layer and/or the cover layer or layers are formed with at least one process from the group of processes, comprising: 3D ink jet printing using STL files, Robocasting of material, e.g. in gel form, stereolithography (SLA), fused deposition (FD) or laminated object manufacturing. It is known from the Korean patent applications KR 1020190075226 A1 and KR 1020170060683 A1 to apply a suspension to tile preforms in a continuous ink-jet process. The suspension has a very low viscosity (approx. 2-20 mPa×s) and is therefore referred to as ink. The problem of the suspension flowing does not play a role here, as the tiles are flat surfaces that are held horizontally when the ink is applied. In addition, the layer thickness is uniform and comparatively thin. Elevated layer structures are not possible. Furthermore, the German patent applications DE 102005023105 A1 and DE 1020055023106 A1 describe the bonding of veneers produced using CAD/CAM to an existing dental framework made of zirconium dioxide. Disadvantages of this teaching are the increased time and material expenditure as well as biological problems arising from the adhesive joints. The patent applications US 2005/0223942 A1 and WO 2016/142234 A1 describe ceramic spraying processes in which ready-to-use mixed ceramic sprays are used. Similar to a paint spray from a can, these ceramic sprays can be applied after shaking. However, due to the propellant in the can, this method cannot be used to apply the ceramic material to a specific point on a dental preform. It should also be noted that, presently, there are 5-axis printers are available in the field of plastics 3D printing, such as the “VSHAPER 5AX” machine from the company VERASHAPE, Tajecina 105, 36-002 Rzeszow-Jasionka, Poland.

### OBJECT OF THE INVENTION

The object of the method, the system and the compound according to the invention is to overcome at least some of the disadvantages of the prior art and to simplify and optimize the manufacturing process of dental prostheses. At the same time, the optical quality of the dental prosthesis is to be improved so that it corresponds more precisely to the natural tooth or teeth to be replaced.

### SUMMARY OF THE INVENTION

According to the invention, a method is proposed for the automated, software-based manufacturing of a veneer of a fixed dental prosthesis, such as a crown or a pontic (bridge), by coating a dental preform with at least one compound which serves to reproduce (imitate) the dentin of a natural tooth. The compound is a ceramic paste formed from a powder of a feldspar ceramic (a glass ceramic) finely dispersed in a solvent. The method comprises the following steps:

a) generating, from image data, a virtual, three-dimensional layer structure which corresponds in shape and size substantially to a spatial volume difference between a first spatial volume and a second spatial volume that is smaller than the first spatial volume, the first spatial volume being the spatial volume of that part of the natural tooth or group of teeth to be replaced by the dental prosthesis, and the second spatial volume being the spatial volume of the preform, the spatial volume difference being intended to be filled with the ceramic paste, wherein the layer structure is divided, in the radial direction, into layers and the layers are divided, in the lateral direction, into volume elements, each volume element being intended to be filled with a portion of the ceramic paste,
b) providing the preform on a carrier that is held by a multi-axis manipulator configured to move and align the preform in space,
c) providing the paste for a print head which is configured to vertically eject individual portions of the paste from a dispensing opening in an ejection direction,
d) coating the preform by filling at least some of the volume elements of the layer structure successively with a single portion of the ceramic paste, comprising, for each volume element, the substeps of:
i. the manipulator aligning the preform in such a way that a ground surface of the volume element to be filled, at an application point on said ground surface, is oriented substantially perpendicular to the ejection direction with the application point lying on the ejection direction,
ii. the print head applying the respective portion of the paste by jerking a valve element in the print head so that the portion is formed and projected in a coherent manner in the direction of the application point and fills the volume element in a punctiform manner,
   e) transporting the coated preform into an oven and
   f) Firing the coated preform in the oven. Furthermore, a system is proposed for the automated, software-based manufacturing of a veneer of a fixed dental prosthesis by coating a dental preform with at least one compound, comprising:
   a computing unit and processing software executable thereon, the software being configured to generate from image data a virtual, three-dimensional layer structure which corresponds in shape and size substantially to a spatial volume difference between a first spatial volume and a second spatial volume that is smaller than the first spatial volume, the first spatial volume being the spatial volume of that part of the natural tooth or group of teeth to be replaced by the dental prosthesis, and the second spatial volume being the spatial volume of the preform, the spatial volume difference being intended to be filled with the ceramic paste, wherein the layer structure is divided, in the radial direction, into layers and the layers are divided, in the lateral direction, into volume elements, each volume element being intended to be filled with a portion of the ceramic paste,
   a carrier designed to hold the preform,
   a multi-axis manipulator that can be moved in space and is designed to hold the carrier,
   at least one print head comprising
a chamber for storing the paste to be ejected, the chamber having a valve opening,
a valve formed by a valve element and the valve opening of the chamber, wherein the valve opening can be sealed by the valve element on the inside of the chamber,
an actuator which is operatively connected to the valve element and is set up to move the valve element jerkily so that individual portions of the paste are formed and ejected from a dispensing opening in an ejection direction, the dispensing opening being the valve opening or an opening spaced apart from it,
whereby the print head is arranged in such a way that the ejection direction is vertical,
   a control unit and a control software executable thereon, the control unit being communicatively connected to the manipulator and the print head and the control software being configured to control the manipulator and the print head depending on the layer structure in such a way that at least some of the volume elements of the layer structure are successively filled with a single portion of the ceramic paste, and in that for each volume element:
the manipulator aligns the preform in such a way that a ground surface of the volume element to be filled, at an application point on said ground surface, is oriented substantially perpendicular to the ejection direction with the application point lying on the ejection direction,
the print head applies the respective portion of the paste by jerking a valve element in the print head so that the portion is formed and projected in a coherent manner in the direction of the application point and fills the volume element in a punctiform manner,
   an oven for firing the coated preform.

Finally, it is also proposed a compound for manufacturing a veneer of a fixed dental prosthesis, which is a ceramic paste formed from a powder of a feldspar ceramic finely dispersed in a solvent and comprising amounts of ammonium polyacrylate, ethoxylated castor oil and traces of urea.

The proposed method and system are destined to replace the manual process of ceramic coating of the preform of a dental prosthesis by an automated application process and to improve it compared to the state of the art. The proposed compound is a ceramic paste that enables the formation of single portions and their punctual application as a whole on the preform, thereby ensuring that the dental prosthesis better matches the natural tooth or teeth to be replaced. The method according to the present invention is a kind of 3D printing method as the veneer is manufactured in that it grows layer by layer.

All known 3D printing methods for manufacturing an object using a ceramic paste are based on a process in which a layer of material is applied on an object carrier with a flat surface by moving a print head along an x axis and an y axis forming an x, y-plane. Once this layer has been “printed”, the object carrier is lowered by one layer thickness in the z-plane and the process starts again. The object to be printed can only be moved in one plane (z-plane) and is otherwise immobile. These known printing processes are basically 2D printing processes, because printing always takes place in only two dimensions, namely in the x, y-plane, apart from the interruptions in the change of the z-axis after completion of a layer. This is the case with all known printing techniques.

The method according to the present invention is a true 3D printing process because the preform can be moved, or moves simultaneously, in all spatial directions x, y, z for the application of the ceramic paste portions in the volume elements of the same layer. More precisely, the preform, by means of the manipulator, can be moved forward, backwards, to the left, to the right, it can be lowered and lifted, relative to the print head. In addition, the preform can also be rotated around an axis. Only in this way it is possible to print on the preform layer by layer which is as a complex shaped body, which is not feasible with conventional methods. This makes the present invention significantly different from all known ceramic printing method.

### EMBODIMENTS OF THE INVENTION

The preform of the dental prosthesis is the supporting structure for the ceramic paste. The preform can be made of a metal or a hard ceramic material. It is the part of the dental prosthesis with which the finished prosthesis is fixed in the mouth. In the case of a crown, this fixation is done on the trunk of the natural tooth to be replaced or on a dental implant, and in the case of a pontic this fixation is done on two or more tooth trunks or two or more dental implants. The ceramic paste, also called slurry, after it has been applied on the preform, forms the veneer of the dental prosthesis, as is generally known in the art. The fully applied ceramic paste is sintered by firing in the oven and then, in the solid state, forms the dental ceramic referred to in the introduction to the invention.

According to a first aspect, the invention proposes a three-dimensional (3D) structure that is determined, purely virtually, by means of the processing software. The 3D-structure defines the geometry, i.e. the shape and size, of the veneer to be manufactured and, respectively, the dental ceramic. The volume of this structure is completely filled with the ceramic paste. Since the desired dental prosthesis should correspond geometrically to the part of the natural tooth or group of teeth to be replaced, the outer geometry of the 3D-structure is that of the part of the natural tooth or group of teeth to be replaced. The inner geometry of the 3D-structure corresponds to the outer geometry of the preform. The shape and size of the 3D-structure therefore corresponds to the spatial volume difference between the first and the second spatial volume, wherein the first spatial volume is the spatial volume of the part of the natural tooth or group of teeth to be replaced by the prosthesis, and the second spatial volume is the spatial volume of the preform which is smaller than the first spatial volume as the preform is part of the prosthesis. The 3D-structure can therefore be obtained subtractively by subtracting the second spatial volume from the first spatial volume.

It is known to determine the geometry, i.e. the shape and size of a dental prosthesis, using a CAD-CAM system (CAD=Computer Aided Design, CAM=Computer Aided Manufacturing) in a dental laboratory. The CAD-CAM software used for this purpose creates a so-called STL file for the design of the dental prosthesis to be produced, in which the surface of the final dental prosthesis is approximated in its shape by triangles. Normally, this STL file is no longer used after the design of the dental prosthesis has been created. It is therefore virtual “waste”. The present invention preferably utilizes this STL file further and thus gains an additional value form the process of CAD-based determination of the shape and size of the dental prosthesis. Since the STL file is already available, the step of determining the outer geometry of the desired dental prosthesis, and of the veneer respectively, is already done. It this therefore advantageous if the processing software uses said STL file to determine the spatial volume difference, and the 3D-structure respectively.

The 3D-structure is then divided, in a radial direction, into layers, to create the 3D layer structure. The thickness of the layers should correspond to the height of a portion of ceramic paste that is projected onto the preform. In this case, all layers share the same height. For example, the thickness can be between 40 μm and 60 μm, preferably 50 μm. Having defined the layer thickness, the number of layers required locally in the structure can be calculated. Here, the word “locally” here refers to a certain application point on the preform and a radial direction, i.e. a directional vector perpendicular to the application point. If the layer structure has a local thickness of 2.5 mm, 50 layers are needed and provided at this application point in the layer structure. If the structure is 1 mm thick, only 20 layers are required. This illustrates that the number of layers varies depending on the thickness of the structure.

Preferably, the STL-file is not only used to determine the spatial volume difference, but also to calculate the amount of locally necessary layers and to generate the layer structure within the spatial volume difference.

When the preform coated with ceramic paste is fired in the oven, the ceramic paste shrinks so that the coated preform loses volume. As a rule, a volume reduction of approx. 15-20% can be expected. It therefore makes sense to take this firing-related shrinkage into account when determining the number of layers in the 3D-structure. This can be done, for example, by providing at least one additional layer in the layer structure, in particular an outer layer, so that the layer structure is larger than the spatial volume difference by this additional layer or layers. In order to distinguish the remaining layers of the layer structure conceptually from the additional layer, the remaining layers are referred to as “regular” layers and the layer thickness created with them is referred to as “regular layer thickness”.

In one embodiment, it may be provided that the at least one additional layer covers the entire preform or completely covers an intermediate regular layer or the outermost regular layer. However, since the degree of shrinkage depends on the thickness of the applied paste, it makes sense to provide one or more additional layers in areas only where the paste is applied thicker on the preform than in the remaining area of the preform. Therefore, in one embodiment, one or more additional layers can be applied in a certain area or areas only depending on the number of layers in these area or areas. In yet another embodiment, at least one additional layer may be provided that covers the entire preform or completely covers an intermediate regular layer or the outermost regular layer and, additionally, one or more additional layer may be provided in areas only, either on the preform, an intermediate regular layer and/or on the outermost regular layer.

For example, for each group with a number x of regular layers, a number y of additional layers can be used in the layer structure. For example, every five or ten (x=5 or x=10) regular layers of the paste, one (y=1) additional layer can be provided in the layer structure. Thus, if the layer structure is at least x regular layers thick in radial direction, y additional layers are provided here, if the thickness is at least 2*x regular layers, 2*y additional layers are provided, and so on.

Once the 3D-structure has been divided into layers, these layers are subdivided, in a lateral direction, into volume elements. In addition, generating the layer structure may comprise determining the geographic coordinates of the application point in each volume element including the normal vector, i.e. the direction perpendicular to the ground of the respective volume element in the application point. This enables the volume element to be aligned with the ejection direction.

In one embodiment, the volume elements can all have the same basic shape. In an alternative, some volume elements have a first basic shape whereas other volume elements have another basic shape. The volume elements can have, with regard to their plan view, a triangular, rectangular, hexagonal or octagonal (honeycomb) basic shape. However, a triangular basic shape of the volume elements is preferred because it allows uneven surface structures to be reproduced more accurately.

It should be noted that the individual layers of the layer structure do not necessarily have to have the same number of volume elements. In other words, one layer may have more or less volume elements than another layer.

In one embodiment, step d), e) and f) of the method can be carried out firstly for only some of the volume elements of the layer structure. This means that, firstly, only a first part of the volume elements is filled with paste and fired, whereas the remaining volume elements stay empty. Afterwards steps d), e) and f) can be repeated for a second part of the volume elements or for all remaining volume elements. Thus, in this embodiment, the coating with the ceramic paste is carried out in stages, which are separated from each other by the step of firing. This has the advantage that intermediate quality checks can be carried out to verify whether the geometry of the preform produced so far corresponds to targeted layer structure which is a kind of construction plan. This will be discussed in more detail below.

In another embodiment, step d) is carried out for all volume elements of the layer structure at once, so that only a single firing step is carried out with step f), i.e. after all volume elements have each been filled with a respective portion of the slurry. This minimizes the manufacturing time for the dental prosthesis, but does not allow for an intermediate quality check.

A second aspect of the method according to the invention is the particular way in which the ceramic paste is applied to the preform. In contrast to the prior art, the ceramic paste is ejected (spit) by the print head onto the preform as individual, coherent portions. In other words, each portion emerges from the dispensing opening of the print head as a single discrete droplet, not as a particle cloud or aerosol. The kinetic movement of the portion of paste is therefore not caused by compressed air.

In order to enable the particular type of portion application according to the present invention, it is proposed that the print head has the chamber for storing the ceramic paste to be ejected and the valve, which is formed from the jerkily movable valve element and the valve opening of the chamber, which the valve element can close from the inside of the chamber. More precisely, the valve element closes the valve opening in a first state and opens it in a second state. For taking the second state, the valve element is lifted away from the valve opening by means of the actuator effecting that the ceramic paste flows out of the chamber and between the valve element and the valve opening. Then the valve element is transferred from the second state to the first state by a jerky movement initiated by the actuator. This has the effect that a part of the ceramic paste that gathered in front of the valve element, between the valve element and the valve opening, is jerkily displaced by the abruptly advancing valve element and pushed through the valve opening out of the chamber. This leads to the generation of a certain amount of ceramic paste. In other words, a certain amount of the ceramic paste is pushed out of the print head jerkingly with high force, which results from the kinetic energy of the valve element's movement and the abrupt volume displacement of the ceramic paste and which both only lasts for microseconds. This makes it possible to apply small amounts of paste to the preform, i.e. portion by portion.

In one embodiment, the dispensing opening of the print head can be the valve opening itself. The certain amount of the ceramic paste ejected from the chamber then directly forms said portion. In another embodiment, the dispensing opening can be an opening spaced apart from the valve opening. For example, an ejection channel of a certain length, for example between on millimeter and a few millimeters in length, can connect to the valve opening and have an opening opposite the valve opening, which forms the dispensing opening of the print head. In other words, said ejection channel is located between the dispensing opening and the valve opening and connects these two openings with each other. The channel fills with paste during operation of the print head and forms a stack, so to speak, which is emptied according to the FIFO principle (First-In-First-Out). This means that a portion that entered the ejection channel first is ejected when the channel is full and further paste is pushed form the chamber into the ejection channel by the valve element. In this case, it is not the part of the paste pushed out of the chamber that forms the said portion, but the part of the paste pushed out of the ejection channel.

The portion ejected from the dispensing opening flies away in the direction of the preform. This direction corresponds to the aforementioned ejection direction. The print head is arranged in that the ejection direction is vertical. More precisely, the trajectory of the portion is not inclined. In normal operation, the trajectory of the portion is quite precise and is almost the same for any other portion. However, slight deviations in target accuracy may occur.

The actuator with which the valve element is operatively connected can be an electromagnetic, a piezoelectric or an air-driven actuator. These kinds of drives enable high acceleration and therefore rapid position changes of the valve element.

This means that the valve element can be forced to move fast forwards, or abruptly (fast acceleration), in the direction of the valve opening, i.e. from the second state into the first state. In contrast, the movement of the valve element from the first state to the second state can be slower or less abrupt than the movement form the second to the first state. The valve element can be designed rod-like in the manner of a piston or plunger. The valve element comprises a head forming a sealing element capable to sealingly close the valve opening.

The ceramic paste can be introduced into the chamber without pressure or with a certain pre-pressure to ensure that it flows between the valve element and the valve opening. It should be emphasized that the pre-pressure is not required to eject the portions from the print head.

The German patent application DE 102019121679 A1 describes a dosing system with a fluidic unit that can be used as the print head described above. DE 102019121679 A1 also describes an actuator unit that can be used to drive the valve element. In addition, the German patent application DE 102020121777 A1 describes a valve element in form of a rod that can be used as the valve element according to the present invention. The international application WO 002014167033A2 describes a pneumatically operated actuator for the same purpose that can be used, in an alternative embodiment, as the actuator according to the present invention. The content of these said applications is hereby incorporated by reference.

The print head is controlled by the control unit, or more precisely by the control software executable on the control unit, in such a way that it triggers the application of a portion of the ceramic paste by the print head as soon as the ground surface of the next volume element to be filled, at the application point on said ground surface, is oriented substantially perpendicular to the ejection direction with the application point lying on the ejection direction. The application point is a target point on the ground of each volume element and in step d) ii. a portion of the slurry is ejected in the direction of this target point.

In a preferred embodiment the application point lies in the centre of the volume element's ground or equals the centre of a circle inscribed within the ground surface, what is, mathematically spoken, the so called “incircle”. The incircle is the biggest circle that can be drawn within a geometric figure. If the geometric figure, and the ground surface respectively, is a triangle, all three edges of that triangle form tangents at the incircle.

It should be noted that the volume element's ground is preferably modelled in the layer structure as a flat plane as it is a basic idea of approximating an arbitrary curved surface by a net of a geometric basic shape like a net of triangles etc. to describe said curved surface by sectionally flat planes bordered by the geometric basic shape. The flat planes and, the volume element's ground respectively, form facets that define the outer shape of the preform or the outer shape of any layer already applied on the preform.

When a portion leaves the dispensing opening, it has a maximum diameter that corresponds to the diameter of the dispensing opening. The dispensing opening can have a diameter of between 200 μm and 500 μm, for example 300 μm. When the portion hits the preform or a layer already applied on the preform, its shape changes from a drop or sphere to a disk or spot. This is because the kinetic energy of the movement of the portion deforms it when the portion hits the preform or the layer already applied on the preform. After impact on the preform the portion forms a spot with an essentially circular shape. In contrast to the prior art, the method according to the invention therefore uses a punctual application of the ceramic paste instead of deposition as particle cloud.

The disk or circular shape of the portion after the impact has a larger diameter than the spherical representation of the portion before the impact, as the mass of the portion is displaced by the impact laterally into its outer periphery. The diameter of a portion applied on the preform is preferably in the range between 300 μm and 700 μm. This is sufficient for the precision of the coating. The diameter can be adjusted using various parameters of the valve, the valve element and the slurry, for example the length of movement (stroke) of the valve element, the movement speed and acceleration of the valve element, the size of the valve opening, and the viscosity of the slurry.

The individual portions of ceramic paste each of which being intended to be filled into one volume element essentially have the same volume or mass. However, in practice, the volume and mass of the individual portions of ceramic paste can vary slightly, for example up to ±20%. This is because, for example, some slurry can adhere at the dispensing opening and can dry out with time.

Each portion applied to the preform can be regarded as a “voxel”, as it not only has the specified extension in lateral direction (diameter), but also a certain height. On average, this height is 50 μm, for example. This makes it possible to calculate, for the layer structure, how many layers must be provided or volume elements must be stacked in order to form the veneer. Preferably, the thickness of the layers in the layer structure is selected to be equal to the height of the applied portions, thus, for example 50 μm.

With regard to the essentially fixed volume or mass of the portions of the ceramic paste it is desirable that the volume elements present a substantially identical volume. This is, however, in practice, hard to achieve when the uneven surface of the preform, and the surface of any layer already applied on the preform is approximated by the volume elements, or more precisely by the volume elements'grounds. Therefore, the volume of the volume elements, or more precisely, due to the fixed height, the ground area of the volume elements may vary from volume element to volume element.

According to step d), a portion of ceramic paste is applied to each volume element. The size of the volume elements can be defined, for example, by setting a maximum and minimum edge length of the borderlines which delimit the ground surface of the volume elements. If the basic shape of the ground surface of the volume elements is a triangle, the three edges delimiting said triangle must have a length between the given maximum and minimum edge length, that is for example between 5 μm and 100 μm.

Generally speaking, the size of the volume elements, or more precisely the lateral dimensions of the volume elements, should be such that the portions (dots or spots) filled in two neighbouring volume elements within the same layer can flow into each other and thus connect materially. The size of a volume element in the lateral direction, i.e. within a layer, is therefore at most so large that a circle of the diameter of an applied portion, for example a circle of 300 μm, can be inscribed in the volume element's ground. In the case of triangular volume elements, this means that the triangles are chosen in that their edges form tangents to an incircle with a maximum diameter of the applied portion.

However, the size of the volume elements should preferably be selected so that the edges delimiting the volume element's ground surface form secants of a circle inscribed within the ground surface with the diameter of the applied portion. This ensures that the individual portions of neighbouring volume elements flow into each other. For example, the size of the ground area of the volume elements can at least be 10% to 20% smaller than the size of the applied portion.

The manipulator effectively forms a robot arm. The manipulator is configured to move the preform, as far as required, along all three spatial axes x, y, z and also to rotate it about at least two, preferably all three, axes of rotation. The manipulator thus has at least five, preferably six degrees of freedom. This serves a third aspect of the invention, which is the alignment of the preform relative to the print head for each application of a portion. More precisely, the preform is aligned by the manipulator in such a way that the ejected portion hits the preform at a right angle in the application point of the volume element to be filled. This ensures that the ground surface of the volume element to be filled lies in a horizontal plane and that the applied ceramic paste does not run down the preform, even if one or more portions of paste have already been applied below the volume element to be filled, i.e. one or more layers of ceramic paste already exist on the preform.

The movement of the manipulator is controlled by the control software. Preferably, the exact geographic position of the preform is determined first. This can be done using a scanner, which has at least two cameras directed at the preform held at the manipulator in order to capture stereoscopic images of the preform. The scanner is connected to the control unit in order to transmit images of the captured preform to the control software. The control software is configured to determine the exact geographic position of the preform relative to a fixed point, e.g. the origin of a cartesian coordinate system. For this purpose, the images captured by the cameras are evaluated by the control software, which determines the geometric position of several reference points on the surface of the uncoated or partially coated preform relative to the scanner, whose position is known in the given cartesian coordinate system having coordinate axis x, y, z. By comparing the reference points with points in the virtual layer structure, the actual position of the preform can be precisely determined. Based on the determined position, the manipulator can be moved to an initial position for the application of the very first portion. For example, this initial position is the position in which the first portion hits a predetermined first volume element at its application point in a right angle. This measure corresponds to a three-dimensional calibration of the initial position of the manipulator.

As an alternative to the cameras, the scanner can be a laser scanner and scan the preform using laser beams by directing laser beams successively at different points on the surface of the uncoated or partially coated preform, and measuring the distance in each case. In this way, the geometric position of several points on the surface of the uncoated or partially coated preform is also determined relative to the scanner, whose position is known in the coordinate system.

The control software calculates the orientation of the preform required for the application of the next portion and controls the manipulator so that the optimum impact angle of the portion projected in the ejection direction is always present. The optimum impact angle is present if the ground surface of the next volume element is essentially horizontally and, therefore, perpendicular to the ejection direction. This alignment of the preform to the print head ensures that the portions always hit the preform at right angles and vertically following the force of gravity. As the preform does not have a flat but complex three-dimensional shape, the manipulator, or more precisely, the software controlling it, ensures that this vertical impact angle is maintained at almost all application points.

Reaching said alignment is, however, difficult in case of a pontic (bridge) in the area between two teeth to be replaced, i.e. the interdental space. This is because vertical application is not easily possible in the interdental spaces. Here, the portion should be applied as far as possible vertically without hitting the ceramic paste already layered on the adjacent tooth. An inclined application angle must be accepted here. The desire for a vertical impact angle applies, above all, to all those volume elements in areas where the layer thickness of the ceramic paste is greatest and where the risk of run-off is high. This is the case for example, at those areas of the veneer that form the incisal edges of an incisor. Here, the surface to be coated with the ceramic paste slopes steeply downwards on both sides and applying a portion at an inclined angle would increase the tendency of the paste to run off.

If the dental prosthesis is a pontic intended to replace the visible parts of two or more natural teeth, the preform consists of a number of main bodies corresponding to the number of teeth to be replaced, which are integrally connected to one another by narrow bridge members. In other words, the preform can have at least two main bodies and a bridge member that is narrower than the main bodies in cross-section and connects the main bodies to each other forming one single piece. The aforementioned problem that a perpendicular impact angle cannot always be ensured exists in particular for those surface areas of the main bodies that face one another. This is the case in the so-called interdental area, i.e. the area that lies between two main bodies and that comprises the bridge member. This is because a part of one main body, which may already be coated with paste, could lie within the ejection direction directed towards the other main body and thus obstruct it. To prevent this problem, it is advantageous to first coat the bridge member between the main bodies with the ceramic paste. This coating includes the coating of the transition area between the bridge member and the main body.

In any case, the method according to the invention is particularly advantageous in the manufacture of a dental prosthesis in the form of a pontic, because the preform intended for the pontic, in contrast to a crown, is at least partially also veneered on the underside.

Filling the volume elements of the layer structure with a portion of the ceramic paste is, in one embodiment, carried out layer by layer. Substeps d)i. and d)ii. are therefore carried out for each volume element of a layer before a volume element of the next layer is filled. In other words, all volume elements of a first layer of the layer structure are filled first, one after the other, and only after that the volume elements of a second layer of the layer structure are filled, with the second layer being radially further out than the first layer. The second layer is therefore formed only when the first layer is completely filled with portions, so that the second layer forms an upper layer and the first layer forms a lower layer. The very first layer is applied to the surface of the preform. However, it is not absolutely necessary for the upper layer to completely cover the lower layer underneath. Due to the necessary geometry of the dental prosthesis, outer layers can only partially cover the underlying layer.

To ensure that the applied portions are circular and do not fray in a star shape like a blob, the distance between the dispensing opening of the print head and the application point of the respective volume element should not exceed a maximum value. The maximum value can be 8 mm or less, for example. The manipulator is controlled in such a way that the maximum value is not exceeded. The minimum distance should be 4 mm. The manipulator is also controlled in such a way that the distance does not fall below the minimum value.

As already mentioned, the volume elements are each filled with a portion of ceramic paste one after the other. The application process on the part of the print head is therefore always discontinuous. In contrast, the movement of the manipulator can be discontinuous or continuous. This is illustrated below.

According to the invention, the manipulator repeatedly realigns the preform in step d)i. in order to position the ground surface of the next, in particular adjacent, volume element at right angles to the ejection direction. Then, in step d)ii. the next portion is applied, i.e. pushed or “shot” from the print head to the preform. The sub-steps d)i. and d)ii. are therefore carried out repeatedly and alternatingly: “align-apply-align-apply-align-apply . . . ” etc.

A discontinuous or stepwise movement of the manipulator exists if the movement is paused each time a portion is applied, i.e. the movement is not continued until the next portion has been applied. In this case, the preform is stationary during sub-step d)ii. i.e. during the application of the portion. In other words, alignment and applying the portion then alternate, which may be the case in one embodiment.

However, the alignment of the preform can also be performed continuously by the manipulator. This means that the manipulator moving the preform, in another embodiment, is not stopped for the execution of sub-step d)ii., but is continued during the execution of sub-step d)ii. In this case, although the ground surface of a volume element is only oriented essentially perpendicular to the ejection direction for a moment, it is readily possible, and proposed by the invention, to coordinate or synchronize the control of the print head and the application of the portions on the one hand, with the control of the manipulator and the movement of the preform on the other hand, in such a way that the application takes place exactly at the moment at which the ground surface is oriented perpendicular to the ejection direction.

A portion can be applied or pushed onto the preform at a low or high cycle frequency. In other words, in sub-step d)ii., a portion can be applied every few seconds or 1, 2 or more portions can be applied per second. For example, the cycle frequency can be between 0.5 Hz and 5 Hz. For example, a new portion can be applied every two seconds or 1, 2, 3, 4 or 5 portions can be applied per second. However, a cycle frequency of up to 20 Hz is also possible.

With such a high cycle frequency, it is not practical to stop the movement for the application of each portion, i.e. x times per second, so that a continuous movement of the manipulator is appropriate here.

In one embodiment, the system comprises a blower that is positioned and directed in such a way that the blower blows heated and/or dehumidified air onto the preform. This has the effect that the applied portions dry more quickly and its flow properties are thereby impaired. This in turn can be used to move the manipulator faster or to increase the cycle frequency for the application of the portions. The blower can have a heater to heat the air. The blower can also have a dehumidifier for dehumidifying. Suitably, the heater and the dehumidifier can be arranged one behind the other in the blower in such a way that air drawn into the blower along a flow path through the blower is first dehumidified in the dehumidifier and then heated in the heater and then exits the blower as dehumidified warm air.

Alternatively or in addition to the blower, an infrared light source can be provided, which is arranged and aligned in such a way that it emits infrared light onto the preform, particularly onto the just applied portions, in order to heat the applied paste locally and thereby dry it.

During operation of the print head, some of the paste may accumulate and dry at the dispensing opening from which each portion leaves the print head. This is one reason why the volume or mass of portions can vary. Furthermore, this accumulated ceramic paste can lead to the ejected portions being deflected and thus not precisely hitting the intended application points or to the portions not forming clean circles on the preform. To avoid this, a cleaning device associated with the dispensing opening can be provided in the system, which is set up and intended to remove accumulated ceramic paste at dispensing opening.

In one embodiment, the cleaning device can, for example, have at least one wing that can be pivoted relative to the dispensing opening. The cleaning device is configured in that in operation, the at least one wing rotates and sweeps over the dispensing opening thereby shearing off accumulated ceramic paste from the dispensing opening. The dispensing opening is thus cleaned and kept clean when the cleaning device is operated regularly. The wing can be lamellar. Furthermore, the wing or at least the part of the wing facing the dispensing opening can be elastic or made of an elastic material so as not to damage the dispensing opening and to effectively wipe off the paste.

The pivoting range of the wing can be limited, for example to a range between 90° and 120°. In other words, the wing does not necessarily have to make full revolutions during operation. However, it is possible for the wing to make full rotations, and it is advantageous to let the cleaning device rotate 360° in case it comprises two or more wings.

The cleaning device can comprise a servo motor and a shaft, the shaft being pivotably or rotatably driven around its axis by the servo motor when actuated. The at least one wing projects from the free end of the shaft that is associated with the dispensing opening and faces away from the servomotor. During operation, the servo motor either swivels the shaft back and forth within an angle defining a swivel range. Alternatively, the servo motor can also rotate the shaft in one direction only.

In one embodiment, the shaft can be arranged parallel to the ejection direction. In this case, the wing is arranged perpendicular to the shaft, so that the wing is oriented at a right angle or transverse to the ejection direction. However, other design variants are also conceivable.

For example, the shaft can be arranged at an angle to the ejection direction, for example at an angle α between 30° and 60°. To ensure that the at least one wing swipes across the dispensing opening in a right angle, in this embodiment, the wing extends away from the shaft at an angle β that is 90° larger than the angle α, wherein angle β relates to the shaft axis in the direction towards the servomotor. For example: if the shaft is arranged at an angle α of 60° to the ejection direction, the wing extends away from the shaft at an angle β of 150° (or 30° in relation to the shaft axis in the direction away from the servomotor). This has the advantage that the end of the shaft can be arranged closer to the dispensing opening and the wing can therefore be shorter, which reduces the bending moment at the free end of the wing opposite the shaft, reduces the risk of vibrations and in addition ensures that the wing sweep the entire dispensing opening.

In one embodiment, the cleaning device can have two or three wings that extend equidistantly from the end of the shaft driven by the servomotor. This improves the cleaning effect. The two or three wings can be designed and arranged identically to the at least one wing, so that reference is made to the above remarks on the at least one wing.

In an advantageous further development, the system can comprise an optical sensor that is arranged in such a way that it detects the dispensing opening and records sensor data associated with the dispensing opening. The sensor is connected to an evaluation unit in order to transmit the sensor data to the evaluation unit. The evaluation unit is set up to evaluate the sensor data to determine whether ceramic paste has accumulated at the dispensing opening and, if this is the case, to trigger a respective control command. More precisely, the evaluation unit triggers the control unit to transmit a control command when the accumulation of ceramic paste becomes too large or exceeds a limit value.

The control command can be, for example, an interruption of the process, a display of a warning message requesting cleaning, and/or the activation of the cleaning device.

The sensor can record sensory data permanently or at intervals and send it to the evaluation unit.

In one embodiment, the optical sensor can be a light barrier that runs directly below the dispensing opening so that the light barrier is interrupted in the event of an accumulation of ceramic paste. An persistent interruption of the light barrier therefore means that ceramic paste has accumulated. The limit value is this case is defined by the distance between the light barrier and the lower edge of the dispensing opening. In one embodiment, the optical sensor can be a camera. The sensory data are then image recordings of the output opening that the sensor creates. The analysis of this image data performed by the evaluation unit can be carried out by comparing the image data with a previously recorded reference image in which the dispensing opening is completely free of ceramic paste. More specifically, the lower edge of the dispensing opening can be monitored in the image data and compared with the lower edge of the dispensing opening in the reference image. If ceramic paste increasingly accumulate at the lower edge of the dispensing opening, the geometry of the edge of the dispensing opening changes over time indicating the need of cleaning.

Preferably, the evaluation unit is part of the control unit.

In one embodiment, the aforementioned scanner, or a further scanner with at least two cameras, can be set up and used to stereographic record images of the partially coated preform or fully coated preform before firing in step f), or of the partially coated preform or fully coated preform after firing in step f). Additionally, an evaluation unit or the previously mentioned evaluation unit can be set up and used to determine reference points on the surface of the coated preform and compare them with the generated layer structure in order to detect deviations in the size and/or geometry of the preform compared to the layer structure. Put simply, the actual and target shapes of the coated preform are compared with each other.

For this comparison, the coated preform is presented to the scanner by the manipulator in several previously defined positions so that the scanner can optically capture the coated preform in different perspectives and an evaluation unit can calculate the outer shape and size of the coated preform from this. To enable the scanner and evaluation unit to determine the curvature of the coated preform, a striped light pattern consisting of alternating light and dark stripes in parallel can be projected onto the coated preform in each of the presented positions. For this purpose, a light source is provided that generates the stripe light pattern, the light source not being located on the same optical axis as the scanner cameras. Based on the distortions of the stripes on the object, the curvature and thus the outer shape and size of the object, i.e. the coated preform can be calculated. This method is generally known in the art but has not yet been used for quality checking in veneering a dental prosthesis. The movements of the coated preform required for scanning are carried out by the manipulator.

The method of checking the outer shape and size of the coated preform can be carried out in between during the coating process in step d), after the coating process in step d) or after the step f), i.e. after firing. If the evaluation unit finds a deviation from the layer structure during the comparison, a corrective measure can be initiated by the control software if necessary. Such a corrective measure can consist of a change to the layer structure, for example by locally adding individual volume elements or adding one or more entire layers.

In order to produce locally different optical and/or mechanical properties within the veneer of the dental prosthesis, it can be provided that the ceramic paste is a first paste with a first optical and/or mechanical property, and at least a second ceramic paste with a different optical and/or mechanical property is provided at and applied by a second print head, which is structurally and functionally identical to the first print head used to dispense the first ceramic paste. The second print head differs from the first print head only in that it dispenses the second ceramic paste that is a slurry as well. Said optical property can be the color, the translucency or the opacity.

In one embodiment, the second paste can be applied to the preform in certain predefined volume elements only, in order to achieve local color nuances in the veneer. In other words, in this case, some of the volume elements of a specific layer are filled with the second ceramic paste and the remaining part of the volume elements of this layer are filled with the first ceramic paste.

Alternatively, the second paste can be applied in layers, i.e. one or more entire layers are formed with the second paste. In this case, all volume elements of a specific layer are filled with the second paste. For example, the preform can initially be coated layer by layer with the first ceramic paste, whereby not every layer has to completely cover the underlying layer in order to form local elevations. The layers formed with the first ceramic paste can be referred to as inner layers. Then, the coated preform is coated further layer by layer with the second ceramic paste. These layers form the outer area of the veneer which is intended for chewing (in case of molars and premolars) or biting (in case of incisors), so that the layers formed with the second ceramic paste can be referred to as outer layers, or more precisely, in the case of incisors as incisal layers and in the case of premolars and molars as occlusal layers.

In one embodiment, the first outer layer covers not only the last inner layer, but also the respective edge area of the other inner layers which protrude from the last inner layer, so that the first outer layer covers the entire preform. This has significant implications on the formation of the layer structure in that the very first layer, and some adjacent layers, do not necessarily cover the whole outer surface of the preform but only a limited area of this preform, wherein the area in its lateral dimension growths from layer to layer so that only one or more of the outer layers cover the whole preform. This way of forming the layer structure also be applied if only one ceramic paste is used.

The combination of the first and second paste help the appearance of the veneer to correspond to the anatomical structure of a natural tooth. Incisors have increased transparency in the anterior region at the level of the incisal edges and in the posterior region at the so-called cusps.

Preferably, both the first ceramic paste and the second ceramic paste contain a glass ceramic powder, but the powders can differ in their amount and/or constitution.

When the first and second ceramic paste is used, the first paste preferably provides the color for the dental prosthesis and a high mechanical strength. The transparency of the first paste is not very high, corresponding to the optical properties of the dentin core of the natural tooth. The average grain size of the ceramic particles of the first paste can be approximately 25 μm. The second paste, in contrast, is characterized by its high transparency after firing and corresponds to the optical properties of the enamel layer of the natural tooth. The so-called glass phase is more concentrated in the powder of the second paste than in the first paste. Furthermore, the average grain size of the ceramic particles of the second paste can be lower than 25 μm.

The first print head and the second print head are preferably arranged adjacent to each other in the system in such a way that the manipulator can position the preform either under the output opening of the first or the second print head. The control software is then set up to control the manipulator in such a way that it aligns the preform according to step d)i. either underneath the dispensing opening of the first or the second print head, depending on the value of a parameter that is assigned to each volume element or at least each layer of the layer structure, wherein the parameter defines if a portion of the first or of the second ceramic paste is to fill the respective volume element. The parameter, therefore, also refers to an optical and/or mechanical property the respective volume element shall have. That is to say, the information defines whether the first paste with the first optical and/or mechanical property or the second paste with the other optical and/or mechanical property is to be used to fill the corresponding volume element. This association between the parameter and the volume elements can be part of a file or a database that is accessed by the control software.

For the skilled person it is obvious that the above concept can be extended to three, four or more ceramic pastes with different optical and/or mechanical properties.

These ceramic pastes will then each be dispensed by a separate print head, to which the manipulator aligns the preform in step d)i. as required or depending on the value of the parameter that is assigned to the corresponding volume element and stored in said file or database.

It should be noted that the ceramic paste according to the invention is applied cold during the method, i.e. it is not heated to influence or reduce its viscosity or to make it workable in the first place.

In order to ensure that the preform is accessible from as many directions as possible in order to be coated with the paste, it is advantageous if the preform is held at a first end of at least one stick. The at least one stick can have a length of between 20 mm and 50 mm, for example. This ensures that there is sufficient free space around the preform to coat it from almost all sides. The at least one stick can have a diameter of between 0.5 mm and 2.5 mm. Preferably, the at least one stick is made of metal. If the dental prosthesis to be produced is a crown, one such stick is sufficient. If the dental prosthesis to be produced is a bridge, two or more of such sticks can be provided.

The other end of the stick or sticks opposite the first end can be embedded or cemented in an embedment compound that is a plastically deformable, curing mass provided in an embedment container. This eases the handling of the preform together with the stick or sticks. The preform, the at least one stick and the embedment container form a simply manageable unit that can easily be attached to the manipulator's carrier. Thus, the preform is not held directly on the manipulator, but indirectly via the at least one stick, and the embedment container. The manipulator then carries the lower part of the unit. The unit can also be removed from the manipulator after coating, transported to the oven, placed in the oven and removed from it again as the stick or sticks as well as the embedment compound and embedment container resists the firing temperature without damage and without dimensional changes.

Ideally, the container should have a flat surface so that the unit can be placed stable on a flat surface.

The container can be held on the manipulator, for example by means of a latching or clamping mechanism, so that quick and tool-free assembly on the manipulator is achieved. Preferably, the manipulator comprises two, three or four clamping jaws between which the container can be clamped. The clamping jaws can be finger-like, for example.

The container can comprise a bottom and a frame enclosing an interior that is at least partially filled by the embedment compound. The embedment compound can be a refractory, initially plastically deformable but curing material in order to insert the holding stick or sticks easily and after curing hold it or them firmly. The material can, for example, be a material typically used for dental casting models made of wax or plastic for the production of crowns, inlays or bridges. Such embedment compounds are known to the skilled person, so that reference is made to the relevant literature.

Preferably, at least one side wall of the frame can be removed for extracting the embedment compound out of the container. For example, said side wall can form a hinge with the bottom or another side wall of the frame so as to swing the side wall away. The side walls forming the frame can be flat, elongated and even, for example made of sheet metal.

According to the invention, a ceramic paste for the manufacture of a veneer of a fixed dental prosthesis is also proposed, and intended to coat a dental preform according to the method according to the invention, whereas the ceramic paste serves to imitate the dentin or dental enamel of a natural tooth.

The invention therefore also relates to a use of the compound according to the invention for the manufacture of a veneer of a fixed dental prosthesis according to the presented method. The compound is characterized in that it is formed from a powder of a feldspar ceramic finely dispersed in a solvent and comprising amounts of ammonium polyacrylate and ethoxylated castor oil and traces of urea.

Due to the combination of the four components mentioned above, i.e. feldspar ceramic, ammonium polyacrylate, ethoxylated castor oil and urea, the ceramic paste is stable with regard to sedimentation for many weeks and can therefore be stored. It can be prepared at any time, regardless of when it is used.

Furthermore, due to its long molecular chains and electrical properties, ammonium polyacrylate is able to attach itself to the negatively charged particles of the ceramic powder. In addition, the ammonium polyacrylate has a network-forming effect in the solvent, that is water for example, so that the molecules of the ammonium polyacrylate cross-link with each other non-covalently through the so-called Van der Waals forces. This also positively influences the flow properties of the ceramic paste.

The ethoxylated castor oil acts as a detergent (surfactant), without which the ammonium polyacrylate and its molecular chains cannot unfold properly. Without the ethoxylated castor oil, entanglements and folding of the molecular chains would occur. The molecules of the ethoxylated castor oil can attach themselves well with their positively charged end to the negatively charged surface of the particles of the ceramic powder. Ideally, the negatively charged ends then protrude centrifugally. This results in a so-called electro-steric inhibition of particle approach which has the effect that the particles of the ceramic powder are kept in suspension and do not stick together.

The viscosity of the paste is between 100 mPa×s and 10,000 mPa×s, preferably between 300 mPa×s and 3,000 mPa×s.

Preferably, the paste has a solids content of approx. 62.5 percent by weight. The solids are formed by the powder of the feldspar ceramic, hereinafter referred to as ceramic powder, and the ammonium polyacrylate, which is also a powder but dissolves completely in the solvent.

The ammonium polyacrylate can have an amount of about 2.6 percent by weight relative to the solids content. The ammonium polyacrylate can have a molecular weight of 3,000-88,000 g/mol, preferably the molecular weight is about 10,000 g/mol. It is a weak electrolyte polymer in a watery solution of about 65%.

The ethoxylated castor oil can have an amount of approx. 1.6 percent by weight relative to the solids content. For example, the solubilizer offered under the brand name “Cremophor EL” from the manufacturer BASF Personal Care and Nutrition GmbH, CM1, Rheinpromenade 1, 40789 Monheim, Germany can be used as ethoxylated castor oil.

Urea is added in about 0,6% by weight relative to the solids content.

Furthermore, the feldspar ceramic powder can have an amount of around 62.5 percent by the total weight of the ceramic paste.

Finally, the solvent can have an amount of about 35.5 percent by weight relative to the total weight of the ceramic paste.

It should be noted that all figures in percent by weight refer to the total weight of the paste. It should also be noted that the weight percentages of the paste ingredients mentioned may vary by +/−10%.

The solvent can be distilled water.

The powder usually consists of particles in the micrometer range. Preferably, the particles are rounded, i.e. not splintered. This significantly improves the flowability without increasing the proportion of solvent in relation to the proportion of powder. It is further preferred that the ceramic particles have a maximum particle diameter of 20 μm. A particle diameter between 3 μm and 5 μm is ideal.

If the initial material of the ceramic powder for producing the paste has a particle size of significantly more than 5 μm, the particle size is reduced by grinding the powder finer in a ball milling process until the final particle size is less than 5 μm, in particular between 3 μm and 5 μm. The grinding process also rounds the particles at the same time. Grinding preferably takes place under vacuum and in a pasty state of the powder in order to prevent the formation of dust and bubbles.

Further features, characteristics, effects and advantages of the invention are explained in more detail hereinafter with reference to examples of embodiments and the accompanying figures. The reference signs contained in the figures retain their meaning from figure to figure. In the figures, reference signs always denote the same or equivalent components, areas, directions or locations as far as specified otherwise.

It should be noted that in the context of the present description the terms “comport”, “comprise” or “include” in no way exclude the presence of other features. Furthermore, the use of the indefinite article for an object does not exclude the plural of said object.

### BRIEF DESCRIPTION OF THE DRAWINGS

FIG. 1: shows a natural healthy tooth

FIG. 2: shows a tooth having a dental prosthesis in form of a crown with a hard-ceramic preform

FIG. 3: shows a tooth having a dental prosthesis in form of a crown with a metallic preform

FIG. 4: visualizes how to determine the shape and size of the veneer needed on the preform

FIG. 5: is enlarged view of detail A in FIG. 4 that shows a part of the cross section of the layer structure for forming the veneer

FIG. 5a: shows a volume element of the layer structure

FIG. 6: shows a perspective view on a layer of the layer structure and the volume elements that constitute the layer

FIG. 7: shows a schematic representation of the system for manufacturing the veneer

FIG. 8a: shows the print head used in the system according to FIG. 7 in more details

FIG. 8b: shows the manipulator used in the system according to FIG. 7 in more details

FIG. 9: shows the print head and an associated cleaning device

FIG. 10: shows the wings of the cleaning device in a view from below

FIG. 11: shows a side view of the cleaning device

FIGS. 12a-12c show a pontic without coating (FIG. 12a), with a disadvantageous coating (FIG. 12b) and with an advantageous coating (FIG. 12c)

FIG. 13: shows a preform in the shape of a pontic held by sticks in an embedment container

FIG. 14: shows a veneer made of two different ceramic pastes for an incisor

FIG. 15: is a flow chart showing the individual steps of the method according to the present invention.

### DETAILED DESCRIPTION OF THE DRAWINGS

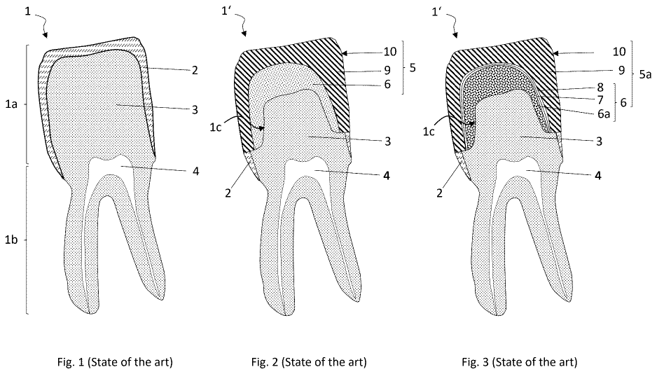

FIGS. 1, 2 and 3 have been explained in the introductory part of the present specification. Reference is made to this introductory part.

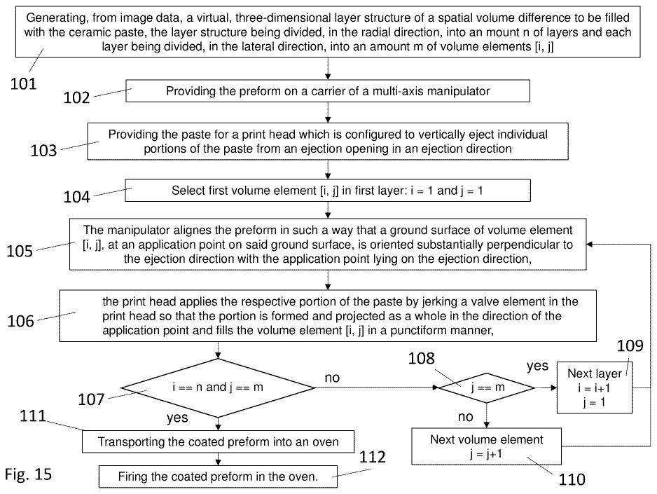

A method for the automated, software-based manufacturing of a veneer 9 of a fixed dental prosthesis 5, 5a, 5b such as a crown 5a, 5b or a pontic 5b (bridge) according to the invention is visualized in FIG. 15 as a flow chart. The veneer 9 is manufactured by coating a dental preform 6, see FIG. 4, with at least one compound which serves to reproduce (imitate) the dentin 3 of a natural tooth 1. The compound is a ceramic paste formed from a powder of a feldspar ceramic finely dispersed in a solvent and comprising amounts of ammonium polyacrylate and ethoxylated castor oil and traces of urea.

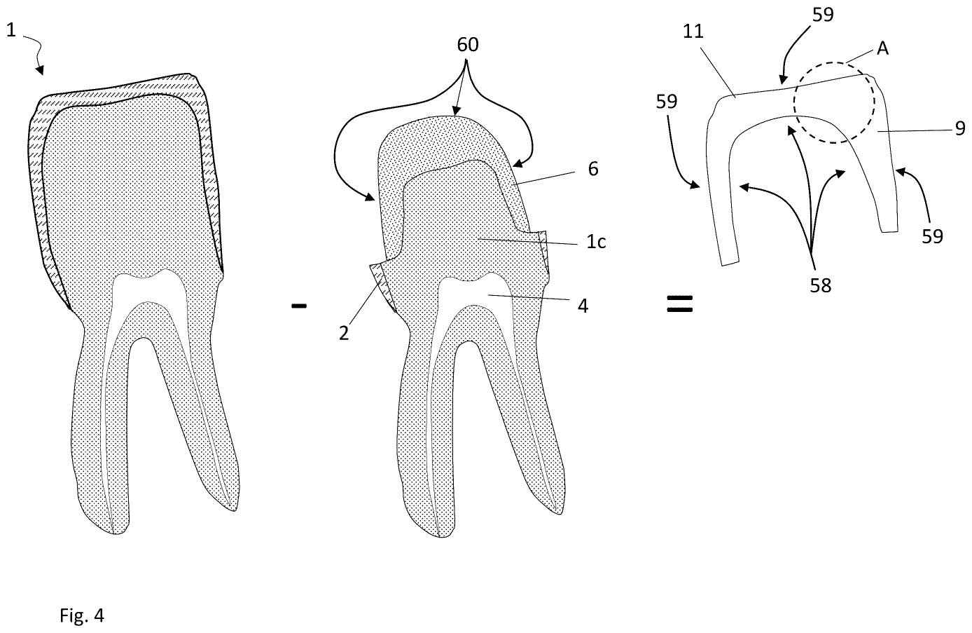

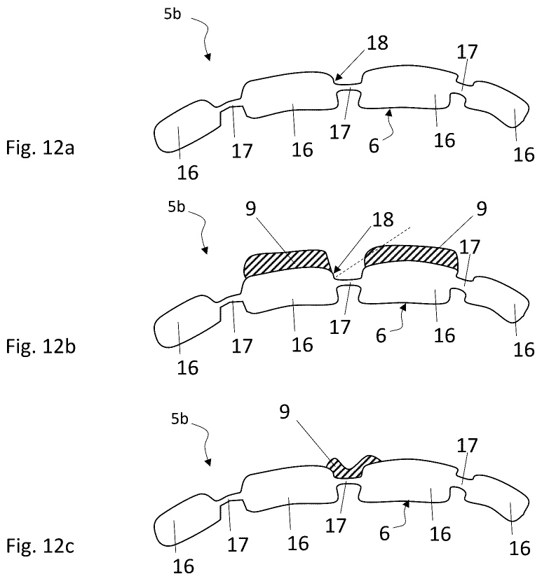

The preform 6 of the dental prosthesis 5, 5a, 5b is the supporting structure for the ceramic paste. The preform 6 is made of a metal or a hard ceramic material. It is the part of the dental prosthesis 5, 5a, 5b with which the finished prosthesis is fixed in the mouth, as shown in FIGS. 2 and 3. In the case of a crown 5, 5a, this fixation is done on the trunk 1c of the natural tooth 1 to be replaced or on a dental implant, and in the case of a pontic 5b (FIGS. 12a-c and 13) this fixation is done on two or more tooth trunks 1c or two or more dental implants. The ceramic paste 26, also called slurry, after it has been applied on the preform 6, forms the veneer 9 of the dental prosthesis 5, 5a, 5b, as is generally known in the art.

In a first step 101 a virtual, three-dimensional (3D) layer structure 11 is generated from image data. This 3D layer structure 11 defines the geometry, i.e. the shape and size, of the veneer 9 to be manufactured and, respectively, of the dental ceramic. As depicted in FIG. 4, the layer structure corresponds in its shape and size substantially to the spatial volume difference between the spatial volume of that part of the natural tooth 1 or group of teeth to be replaced by the dental prosthesis 5, 5a, 5b (first spatial volume) and the spatial volume of the preform 6 (second spatial volume). This is visualized in FIG. 4 by an equation.

FIG. 4 shows, on the left side, the shape of a natural tooth 1 to be reproduced and, respectively, the first spatial volume that is the spatial volume taken by the natural tooth 1. This shape is the target when manufacturing the veneer 9. In the centre of FIG. 4 the tooth 1 is given with removed head 1a that needs to be replaced by a crown 5a. There is only left a tooth trunk 1c on which the preform 6 is already positioned. The second spatial volume is the spatial volume of the preform 6. As can be seen from the mathematic equation that is visualized by FIG. 4, the part of the crown 5a that is missing in the middle picture can be obtained by subtracting the spatial volume of the preform 6 from the spatial volume of the natural tooth 1 to be reproduced. Obviously, the second spatial volume is smaller than the first spatial volume. The result of this mathematic calculation, i.e. the spatial volume difference between the first and second spatial volume is visualized in the right picture in FIG. 4. Since the desired dental prosthesis 5, 5a, 5b must correspond geometrically to the part of the natural tooth 1 or group of teeth to be replaced, the outer geometry 59 of the layer structure 11 is that of the part of the natural tooth 1 or group of teeth to be replaced. The inner geometry 58 of the layer structure 11 corresponds to the outer geometry 60 of the preform 6. The 3D-structure is therefore be obtained subtractively by subtracting the second spatial volume from the first spatial volume. The spatial volume difference is intended to be filled with the ceramic paste which then forms the veneer 9.

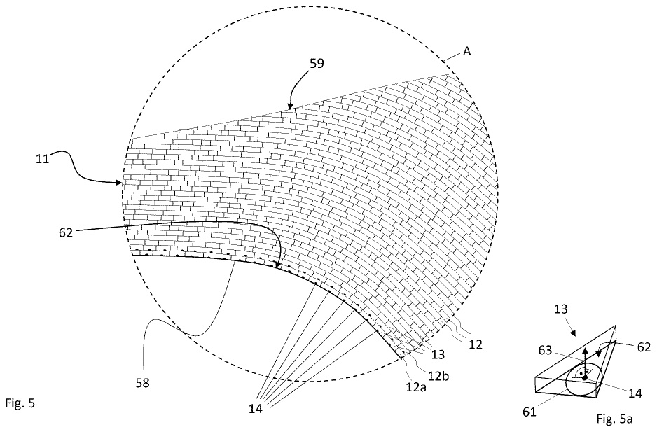

In order to systematically fill the spatial volume difference with the ceramic paste in an automated, software-based manner, the layer structure in the spatial volume difference is determined. This is done by dividing the spatial volume difference into n layers 12 and each layer into mi volume elements 13, as can be seen in FIG. 5. This results in the layer structure 11 being divided, in the radial direction, into the layers 12 and the layers being divided, in the lateral direction, into the volume elements 13. Each volume element 13 is now intended to be filled with a portion of the ceramic paste. This is done as follows:

In step 102 in FIG. 5 the preform 6 is provided on a carrier 40 that is held by a multi-axis manipulator 21 configured to move and align the preform 6 in space.

In step 103 the ceramic paste is provided for a print head 22 which is configured to vertically eject individual portions 19 of the paste from a dispensing opening 27a in an ejection direction R.

In step 104 a first volume element 13 to be filled is selected. To identify the volume elements 13 each volume element 13 is associated with a first index i and a second index j. The first index i defines the layer 12 of which the volume element 13 is part, the second index j is a number that identifies the respective volume element 13 within the respective layer 12, so that the combination of the two indexes [i, j] unambiguously defines a certain volume element 13. Selecting the first volume element in step 104 means, for example, setting the indexes i=1 (first layer) and j=1 (first volume element).

Now coating the preform 6 takes place by filling at least some of the volume elements 13 of the layer structure 11 successively with a single portion 19 of the ceramic paste. For that, steps 105 and 106 are repeated for each volume element 13 that is to be filled.

In step 105 the manipulator 22 aligns the preform 6 in such a way that the ground surface 62 of the volume element 13 to be filled, at an application point 14 on said ground surface 62, is oriented substantially perpendicular to the ejection direction R with the application point 14 lying on the ejection direction R. Then, in step 106, the print head 22 applies the respective portion 19 of the paste by jerking a valve element 52 in the print head 22 2 so that the portion 19 is formed and projected in a coherent manner in the direction of the application point 14 and fills the volume element 14 in a punctiform manner.

After a volume element 13 is filled, the next volume element 13 is selected. This is done by increasing the index j in step 110 or, if all volume elements 13 of a layer 12 have been filled (see step 108), by increasing the index i in step 109 (go to the next layer 12) and resetting the index i to 1 (begin with the first volume element). Thus, filling the volume elements 13 of the layer structure 11 with a portion 19 of the ceramic paste 26 is carried out layer by layer. In other words, all volume elements 13 of a first layer 12 of the layer structure 11 are filled first, one after the other, and only after that the volume elements 13 of a second layer 12 of the layer structure 11 are filled, with the second layer being radially further out than the first layer. The second layer is therefore formed only when the first layer is completely filled with portions, so that the second layer forms an upper layer and the first layer forms a lower layer. The very first layer is applied to the surface of the preform 6 whereas every further layer 12 is applied on a previous layer 12.

In step 107 it is checked if the indexes i, j have reached their maximum, i.e. all volume elements 13 of all layers 12 have been filled. If this is the case, the coated preform is transported into an oven, step 111, and fired therein, step 112. During firing the ceramic paste is sintered and becomes solid thereby forming a dental ceramic 9 and the veneer of the dental prosthesis 5, 5a, 5b.

Note that the flow chart in FIG. 5 is simplified for the sake of understanding. Several aspects are not reflected in FIG. 5. For example, it is supposed in FIG. 15 that all layers 12 have the same number m of volume elements 13 which is not true. The number of volume elements 13 differs from layer 12 to layer, so that rather a layer specific index mi shall be used, defining, for each layer 12 i, the number mi of volume element 13 in this layer 12 i.

Furthermore, it is supposed in FIG. 15 that all volume elements 13 of all layers 12 are filled with a portion of the ceramic paste before step 112 is executed, i.e. the fully coated preform is fired in the oven. However, in another embodiment, only some volume element 13 are filled with portions of the ceramic paste and step 112 is executed for these filled volume elements, i.e. a partially coated preform is fired firstly, and then some other or all remaining volume elements 13 are filled with portions of the ceramic paste and step 112 is executed again for these other or the remaining filled volume elements 13. In this case the step of firing 112 is performed twice or even more time. This makes it possible to introduce intermediate checks whether the coating is on schedule, i.e. corresponds to the layer structure 11.

Due to repetition of steps 105 and 106 the manipulator 21 and the print head 22 are controlled to alternatingly carry out the steps: “align-apply-align-apply-align-apply . . . ” etc. Thus, a discontinuous or stepwise movement of the manipulator 21 is used and the movement of the manipulator 21 and preform 6 is paused each time a portion is applied. However, the alignment of the preform 6 can also be performed continuously by the manipulator. This means that the manipulator 21 moving the preform 6, in another embodiment, is not stopped for the execution of step 106, but is continued during the execution of step 105. In this case, the control of the print head 22 and application of the portions 19 on one hand is synchronized with the control of the manipulator 21 and the movement of the preform 6 on the other hand, namely in such a way that the application of a portion 19 takes place exactly at the moment when the ground surface 62 is perpendicular to the ejection direction R with the application point lying on the ejection direction R.

Preferably, a portion 19 is applied or pushed onto the preform 6 several times per second. The number of times per second defining a cycle frequency. This cycle frequency can be between 0.5 Hz and 5 Hz, whereas a cycle frequency of 0.5 Hz means that a portion 19 is applied every two seconds.

With regard to the layer structure 11 illustrated in FIG. 5 an upper layer 12 does not necessarily cover a lower layer 12 completely. Due to the necessary geometry of the dental prosthesis, outer layers 12 can only partially cover the underlying layer 12. This however, is a matter of generating the layer structure 1. The layer structure in FIG. 5 is constructed from the inside outwards, beginning at the inner geometry 58 of the spatial volume difference. However, alternatively, you can construct the layer structure 11 by defining an outmost layer first that extends across and defines the whole outer geometry 59 of the layer structure 11, so that this outmost layer covers the inner layers and the preform 6 completely. In this case, the layer structure 11 is constructed from the outside inwards, beginning at the outer geometry 59 of the spatial volume difference. This alternative is advantageous if two or more ceramic pastes are used, one of which shall be applied in volume elements 13 of the outer layers and the other shall be applied in volume elements 13 of the inner layers.

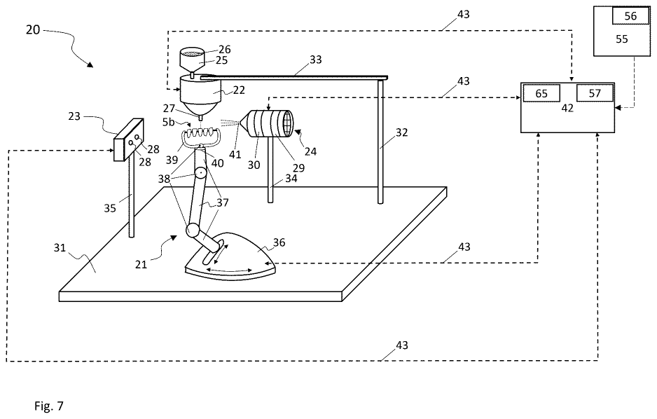

Now turning to FIG. 7, a system 20 is described for the automated, software-based manufacturing of the veneer 9 of a fixed dental prosthesis 5, 5a, 5b by coating the dental preform 6 with the at least one compound. Basically, the system 20 comprises at least a computing unit 55, a carrier 40, a manipulator 21, at least one print head 22 and a control unit 42, the basic functions of which are explained in the following. Said components are arranged on a base plate 31. The system 20 further comprises an oven for firing the coated (partially or full) preform 6. However, this oven is omitted in FIG. 7.

The computing unit 55 comprises a processing software 56 that is executed on the computing unit 55 and is configured to generate from image data the virtual, 3D layer structure 11. For that, a so called STL file is used containing image data of the tooth head to be replaced by the dental prosthesis 5, 5a, 5b. From that file the software 56 calculates the spatial volume difference, divides it, in the radial direction, into the layers 12 and divides the layers 12, in the lateral direction, into the volume elements 13. The term “radial direction” here means from the preform 6 outwards, and the term “lateral direction” refers to the dimensions of the layers 12.

The thickness of the layers 12 correspond to the height of a portion 19 of ceramic paste 26 after it hit the preform 6. All layers 12 share the same height that is set here to approximately 50 μm.

Generating the layer structure 11 additionally comprise determining the geographic coordinates of the application point 14 in each volume element 13 including the normal vector 63, i.e. the direction perpendicular to the ground 62 of the respective volume element 13 in the application point 14, see FIG. 5a. This enables the volume elements 13 to be aligned with the ejection direction R. The geographic coordinates are given, for example, by x, y, z coordinates in a cartesian coordinate system with coordinate axis x, y, and z.

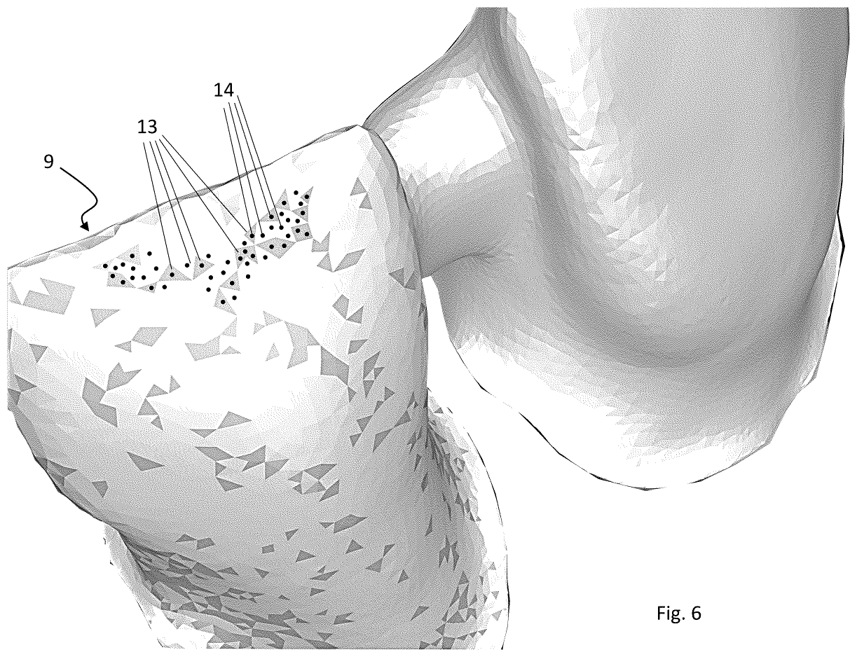

As can be seen in FIG. 6 the presented outermost layer 12 of the veneer 9, all volume elements 13 of that layer 12 have the same basic shape that is, with regard to their plan view, a triangle. To put it more simply, the volume elements 13 are triangles. They have a plane ground 62 each so as to form a facet of the respective layer 12. FIG. 5a shows a single triangular volume element 13 with even ground 62 and constant height.

The carrier 40 is designed to directly or indirectly hold the preform 6, and the manipulator 21 is a multi-axis manipulator 21 that can be moved in space and is designed to hold the carrier 40 and to move and align the preform 6 respectively.

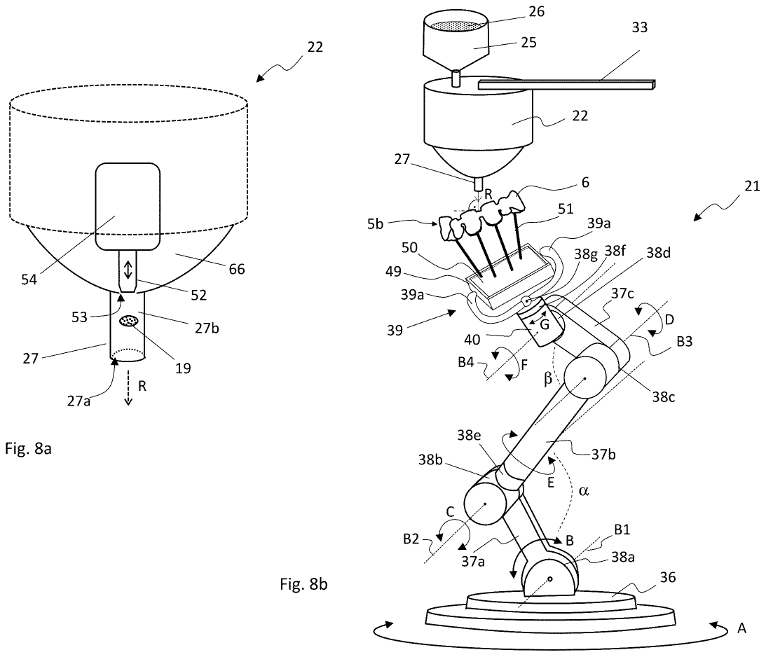

The print head 22 is fixed at a supporting arm 33 that protrudes from a mounting rod 32. The mounting rod 32 is mounted on the base plate 31. The print head 22 comprises, in its interior, a chamber 66 for storing the ceramic paste 26 before ejection. The ceramic paste 26 is provided in a container 25 attached outside to the print head 22. As can better be seen from FIG. 8a the chamber 66 comprises a valve opening 53 and a valve that is arranged inside the chamber 66. The valve comprises a valve element 52, an actuator 54 and the valve opening 53, wherein the valve opening 53 can be sealed by the valve element 52 on the inside of the chamber 66. The valve element 52 closes the valve opening 53 in a first state and opens it in a second state. The actuator 54 is operatively connected to the valve element 52.

For taking the second state, the valve element 52 is lifted away from the valve opening 53 by means of the actuator 54 effecting that the ceramic paste 26 flows out of the chamber 66 and between the valve element 52 and the valve opening 53. Then the valve element 52 is moved from the second state to the first state by a jerky movement initiated by the actuator 54. In other words, the actuator 54 is set up to move the valve element 52 jerkily, i.e. with high acceleration. This has the effect that a part of the ceramic paste 26 that gathered in front of the valve element 52, between the valve element 52 and the valve opening 53, is jerkily displaced by the abruptly advancing valve element 52 and pushed through the valve opening 53 out of the chamber 66. This leads to the generation of a certain amount of ceramic paste 26, a portion 19. In other words, a portion 19 of the ceramic paste 26 is ejected jerkingly with high force from a dispensing opening 27a in the ejection direction R which results from the kinetic energy of the valve element's movement and the abrupt volume displacement of the ceramic paste 26 and which both only lasts for microseconds. This makes it possible to apply small amounts of ceramic paste to the preform 6, i.e. portion by portion.

Each portion 19 emerges from the dispensing opening 27a of the print head 22 as a single discrete droplet. Here, the dispensing opening 27a is spaced from the valve opening 53 by an ejection channel 27b. As can further be seen in FIGS. 7, 8a and 8b, the print head 22 is arranged in such a way that the ejection direction R is vertical.

The manipulator 21 is controlled in such a way that the distance between the dispensing opening 27a and the application point 14 of the respective volume element 13 does not exceed a maximum value of 8 mm. The manipulator 21 is also controlled in such a way that the distance does not fall below a minimum value of 4 mm.

The actuator 54 is an electromagnetic, a piezoelectric or an air-driven actuator. These kinds of drives enable high acceleration and therefore rapid position changes of the valve element 52. This means that the valve element 52 can be forced to move fast forwards, or abruptly (fast acceleration), in the direction of the valve opening 53, i.e. from the second state into the first state. In contrast, the movement of the valve element 52 from the first state to the second state can be slower or less abrupt than the movement form the second to the first state. The valve element 52 is designed rod-like in the manner of a piston or plunger. The valve element 52 comprises a head forming a sealing element capable to sealingly close the valve opening 53.

The control unit 42 comprises a control software 57 that is executed on the control unit 42. The control unit 42 is connected to the manipulator 21 and the print head 22 by means of wired or wireless control lines 43. The control software 57 is configured to control the manipulator 21 and the print head 22 depending on the layer structure 11 in such a way that at least some of the volume elements 13 of the layer structure 11 are successively filled with a single portion 19 of the ceramic paste 26. More precisely the control software 57 controls the manipulator 21 in that it aligns the preform 6 in such a way relative to the print head 22 that the ground surface 62 of the volume element 13 currently to be filled, at an application point 14 on said ground surface 62, is oriented substantially perpendicular to the ejection direction R with the application point 14 lying on the ejection direction R. Furthermore, the control software 57 controls the print head 22 in that it applies the respective portion 19 of the paste 26 by jerking the valve element 52 in the print head 22 so that the portion 19 is formed and projected in a coherent manner in the direction of the application point 14 and fills the volume element 13 in a punctiform manner.

The control software 57 triggers the application of a portion 19 of the ceramic paste by the print head 22 as soon as the ground surface 62 of the next volume element 13 to be filled, at the application point 14 on said ground surface 62, is oriented substantially perpendicular to the ejection direction with the application point 14 lying on the ejection direction R. The application point 14 is a target point on the ground 62 of each volume element 13 and in step 106 a portion 19 of the slurry 26 is ejected in the direction of this target point.

As can be seen in FIG. 5a, in this embodiment, the application point 14 equals the centre of a circle 61 inscribed within the ground surface 62, what is, mathematically spoken, the so called “incircle” 61. The incircle 61 is the biggest circle that can be drawn within a geometric figure. If the volume elements 13, and their ground surface 62 respectively, are triangles as in FIGS. 5, 5a and 6, all three edges of that triangle form tangents at the incircle 16, see FIG. 5a.

When a portion 19 leaves the dispensing opening 27a (FIG. 8a), it has a maximum diameter that corresponds to the diameter of the dispensing opening 27a. The dispensing opening 27a has a diameter of approximately 300 μm. When the portion 19 hits the preform 6 or a layer 12 already applied on the preform 6, the portion's shape changes from a drop or sphere to a disk or spot. This is because the kinetic energy of the movement of the portion 19 deforms it when the portion 19 hits the preform 6 or the layer 12 already applied on the preform 6. After impact on the preform 6 the portion 19 forms a spot with an essentially circular shape. In contrast to the prior art, the method according to the invention therefore uses a punctual application of the ceramic paste 26 instead of painting or deposition as particle cloud.

The disk or circular shape of the portion 19 after the impact has a larger diameter than the spherical representation of the portion 19 before the impact, as the mass of the portion 19 is displaced by the impact laterally into its outer periphery. The diameter of a portion 19 applied on the preform 6 (after impact) is preferably between 400 μm and 500 μm.

Knowing this diameter or having defined this diameter, the size of the volume elements 13 can be set such that the portions 19 filled in two neighbouring volume elements 13 within the same layer 12 can flow into each other and thus connect materially. The size of a volume element 13 in the lateral direction, i.e. within a layer 12, is therefore chosen in that a circle of the diameter of an applied portion 19 (after impact), for example a circle of 400 μm, extends beyond the edges bordering the ground 62, which is the case if the incircle 61 has a diameter smaller than the diameter of an applied portion 19, for example 300 μm only. If the size of the volume elements 13 is defined by setting a maximum and minimum edge length, this edge length can be for example between 5 μm and 100 μm to ensure that the applied portion 19 (after impact) extends beyond the edges.

The manipulator 21 effectively forms a robot arm. The manipulator 21 is configured to move the preform 6, as far as required, along all three spatial axes x, y, z and also to rotate it about all three axes of rotation. The manipulator 21 thus has six degrees of freedom. This enables to exactly align the preform 6 relative to the print head 22 for each application of a portion 19. More precisely, the manipulator 21 aligns the preform 6 in such a way that the ejected portion 19 hits the preform 6 at a right angle in the application point 14 of the volume element 13 to be filled. In this aligned state the ground surface 62 of the volume element 13 to be filled lies in a horizontal plane so that the applied portion 19 does not run away.

FIG. 8b shows the manipulator 21 in more details. The manipulator 21 comprises a foot 36 that can be rotated around a vertical axis. This is illustrated by a curved arrow

A. The foot 36 rests on a pivot bearing by means of which the whole manipulator 21 can be oriented in a certain angular position. The manipulator 21 further comprises multi-section arm 37 with several pivot bearings 38. In Particular, the manipulator 21 comprises a lower arm 37a, a middle arm 37b and an upper arm 37c as well as a first pivot bearing 38a, a second pivot bearing 38b, a third pivot bearing 38c and a fourth pivot bearing 38d.

The lower end of the lower arm 37a is pivotably mounted on the foot 36 by means of the first pivot bearing 38a that enables to swivel the lower arm 37a around a first horizontal axis B1. This is illustrated by a curved arrow B1.

The upper end of the lower arm 37a is pivotably connected to the lower end of the middle arm 37b by means of the second pivot bearing 38b. This enables to adjust the angle α between the lower arm 37a and the middle arm 37b by swivelling the middle arm 37b around a second horizontal axis B2 of the second pivot bearing 38b. This is illustrated by a curved arrow C.

The upper end of the middle arm 37b is pivotably connected to the first end of the upper arm 37c by means of the third pivot bearing 38c. This enables to adjust the angle β between the middle arm 37b and the upper arm 37c by swivelling the upper arm 37c around a third axis B3 of the third pivot bearing 38c. This is illustrated by a curved arrow D.

Additionally, the middle arm 37b comprises a further pivot bearing 38e enabling to rotate an upper section of the middle arm 37b around its longitudinal axis relative to a lower section of the middle arm 37b that is fixed at the second pivot bearing 38b. This is illustrated by a curved arrow E. in consequence, the third axis B3 can also be swivelled around the longitudinal axis of the middle arm 37b.

The second end of the upper arm 37c comprises a fourth pivot bearing 38d to which the carrier 40 is connected so as to swivel the carrier 40 around a fourth axis B4 of the fourth pivot bearing 38d. This is illustrated by a curved arrow F. A further pivot bearing 38 similar to that of the middle arm 37b can be arranged in the upper arm 37c

The carrier 40 comprises a connecting portion to which a clamping or gripping mechanism 39 is connected. The connecting portion can be rotated, by means of a further pivot bearing 38f, relative to that part of the carrier 40 mounted at the fourth pivot bearing 38d. This is illustrated by a curved arrow G.

The clamping or gripping mechanism 39 enables a quick and tool-free assembly of the preform 6 on the manipulator 21. Here, the mechanism 39 comprises two opposing clamping jaws 39a, 39b that are pivotable around a common axis 38g. The clamping jaws 39a, 39b can be swung apart against a spring force that biases the clamping jaws 39a, 39b and provides that they firmly hold an object placed between them. In the embodiment shown in FIGS. 7 and 8b the clamping jaws are finger-like. This, however, is a pure example, as many other ways of realizing how the preform 6 is held at the manipulator 21 are possible.

The movement of the manipulator 21 is controlled by the control software 57. Each pivot bearing 38 can be actuated by a respective stepping motor, for example, setting the angular position of the respective pivot bearing 38.

For correct movement of the preform 6, the exact geographic position of the preform 6 is determined first. This is done using a scanner 23, which has at least two cameras 28 directed at the preform 6 in order to capture stereoscopic images of the preform 6. The scanner 23 is arranged on a mounting rod 35 that is mounted on the base plate 31 as well. The scanner 23 is connected to the control unit 42 by a control line 43 in order to transmit images of the captured preform 6 to the control software 57. The control software 57 is configured to determine the exact geographic position of the preform 6 relative to a fixed point, e.g. the origin of a cartesian coordinate system or the position if the scanner 23. Images captured by the cameras 28 are evaluated by the control software 57, which determines the geometric position of several reference points on the surface of the uncoated or partially coated preform 6 relative to the scanner 23, for example, whose position is known in the given cartesian coordinate system having coordinate axis x, y, z.

By comparing the reference points with points in the virtual layer structure 11, the actual position of the preform 6 can be precisely determined. Based on the determined position, the manipulator 21 can be moved to an initial position for the application of the very first portion 19 in the first volume element 13 (j=1) of the first layer 12 (i=1). The initial position is the position in which the first portion 19 hits the first volume element 13 at its application point 14 in a right angle.

The control software 57 calculates the necessary orientation of the preform 6 required for the application of the next portion 19 and controls the manipulator 21 so that, on one hand, the ground surface 62 of the next volume element 13 is essentially horizontally and, therefore, perpendicular to the ejection direction R, and, on the other hand, that the ejection direction R runs through the application point 14. This effects that the portion 19 hits the volume element 13 in the application point 14 in a perpendicular impact angle.

If the dental prosthesis 5, 5b is a pontic 5b intended to replace the visible parts of two or more natural teeth 1, the preform 6 consists of a number of main bodies 16 corresponding to the number of teeth to be replaced, and one or more narrow bridge members 17 that integrally connect two main bodies 16 with each other forming one single piece. Turning to FIGS. 12a-c the preform 6 shown therein comprises four main bodies 16 and three bridge members 17 each of which is narrower than the connecting main bodies 16 in cross-section. FIG. 12a illustrates an uncoated preform 6.

In those surface areas of the main bodies 16 that face one another a perpendicular impact angle cannot always be ensured. This is the case in the so-called interdental area, i.e. the area that lies between two main bodies 16 and that comprises the respective bridge member 17. This is because a part of one main body 16, which is already coated with ceramic paste 9 as shown in FIG. 12b, could lie within the ejection direction directed towards the other main body 16 and thus obstruct it. This is illustrated in FIG. 12b by a dotted line that ends at an inner edge 18 where the bridge member 17 merges into the main body 16. To prevent this problem, it is envisaged to first coat the bridge member 17 between the main bodies 16 with the ceramic paste 9. This coating includes the coating of the transition area, i.e. the inner edge 18 between the bridge member 17 and the main body 16, as shown in FIG. 12c.

As depicted in FIG. 7 the system 20 also comprises a blower 24 that is positioned on a mounting rod 34 and directed to the preform 6. The blower 24 blows heated and/or dehumidified air onto the preform 6. This has the effect that the applied portions 19 dry more quickly and their flow properties are thereby impaired. The blower 24 comprise a heater 30 to heat the air, and a dehumidifier 29 for dehumidifying. The heater 30 and the dehumidifier 29 are arranged one behind the other in the blower 24 in such a way that air drawn into the blower 24 along a flow path through the blower 24 is first dehumidified in the dehumidifier 29 and then heated in the heater 30 and then exits the blower 24 as dehumidified warm air.

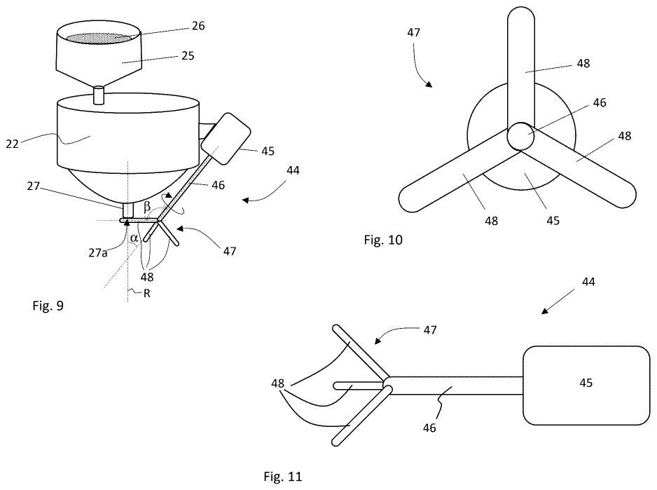

During operation of the print head 22, some of the paste 26 may accumulate and dry at the dispensing opening 27a out of which each portion 19 leaves the print head 22. In order to keep the dispensing opening 27a free of accumulated paste 26, a cleaning device 44 is associated with the dispensing opening 27a. This cleaning device 44 is shown in FIGS. 9, 10 and 11 in different view. FIG. 9 shows the cleaning device 44 associated with the print head 11, FIG. 10 shows the cleaning device 44 from below and FIG. 11 is a side view of the cleaning device 44.

The cleaning device 44 comprises a propeller 47 with three lamellar-like wings 48, a shaft 46 from which the wings 48 project equidistantly, and a servo motor 45 that rotates the shaft 46 when actuated around the shaft axis. As is apparent from FIG. 11, the wings 48 project from the free end of the shaft 46 at an angle β of approximately 120° facing away from the servomotor 45, see FIG. 9. Said free end is associated with the dispensing opening 27a.

The cleaning device 44 is arranged, relative to the print head 22, such that the shaft 46 lies in an angle α of approximately 30° to the ejection direction R. Thus, the wings 48 pass the dispensing opening 27a in a right angle. In operation, the wings rotate and sweeps over the dispensing opening 27a thereby shearing off accumulated ceramic paste 26 from the dispensing opening 27a. The dispensing opening 27a is thus cleaned and kept clean is the cleaning device 44 is operated regularly. T

he wings 48 or at least the part of the wing 48 facing the dispensing opening 27a are elastic or made of an elastic material, like rubber, so as not to damage the dispensing opening 27a and to effectively wipe off the paste 26.

The system 20 can comprise an optical sensor that is arranged in such a way that it detects the dispensing opening 27a and records sensor data associated with the dispensing opening 27a. The optical sensor is connected to an evaluation unit 65 in order to transmit the sensor data to the evaluation unit 65. The evaluation unit 65 is part of the control unit 42 and set up to evaluate the sensor data to determine whether ceramic paste 26 has accumulated at the dispensing opening and, if this is the case, to trigger a respective control command. More precisely, the evaluation unit 65 triggers the control unit 42 to transmit a control command when the accumulation of ceramic paste becomes too large or exceeds a limit value. The control command is an activation of the cleaning device 44 and, if the control command is issued during the method of applying the portions, an interruption of the process.

In one embodiment, the optical sensor can be a light barrier that runs directly below the dispensing opening 27a so that the light barrier is interrupted in the event of an accumulation of ceramic paste 26. An persistent interruption of the light barrier therefore means that ceramic paste 26 has accumulated.

Alternatively, the optical sensor can be a camera. The sensory data are then image recordings of the output opening 27a that the sensor creates. The analysis of this image data performed by the evaluation unit 65 can be carried out by comparing the image data with a previously recorded reference image in which the dispensing opening 27a is completely free of ceramic paste 26. More specifically, the lower edge of the dispensing opening 27a can be monitored in the image data and compared with the lower edge of the dispensing opening 27a in the reference image. If ceramic paste increasingly accumulates at the lower edge of the dispensing opening 27a, the geometry of the edge of the dispensing opening 27a changes over time indicating the need of cleaning.

As previously mentioned, intermediate checks whether the coating is on schedule, i.e. corresponds to the layer structure 11 can take place. For this comparison, the coated preform 6 is presented to the scanner 23 by the manipulator 21 in several previously defined positions so that the scanner 23 can optically capture the coated preform 6 in different perspectives and an evaluation unit 65 can calculate from this the outer shape and size of the preform 6 coated so far. To enable the scanner 23 and evaluation unit 65 to determine the curvature of the coated preform 6, a striped light pattern consisting of alternating light and dark stripes in parallel is projected onto the coated preform 6 in each of the presented positions. For this purpose, a light source (not shown in FIG. 7) is provided that generates the stripe light pattern, the light source not being located on the same optical axis as the scanner's cameras 28. Based on the distortions of the stripes on the object, the curvature and thus the outer shape and size of the coated preform 6 can be calculated and compared to the layer structure 11 to determine deviations.

The method of checking the outer shape and size of the coated preform 6 can be carried out in between during the coating process in steps 105 and 106, after the coating process in 106 or after step 112 (firing). If the evaluation unit finds a deviation of the curvature from the layer structure 11 during the comparison, a corrective measure is initiated by the control software. Such a corrective measure is for example adding individual volume elements 13 or adding one or more layers 12.

In order to produce locally different optical and/or mechanical properties within the veneer 9 of the dental prosthesis 5, 5a, 5b, a first and a second ceramic paste 26, 26a is used that have different optical and/or mechanical properties. Said optical property can be the color, the translucency or the opacity. The system 20, in this case, comprises a first print head and a second print head both of which being identical in structure and function to the print head 22 described above in relation to FIGS. 7, 8 and 8b. The first ceramic paste 26 is applied by the first print head and the second ceramic paste 26a is applied by the second print head.

The second paste 26a is applied to the preform 6 in certain predefined volume elements 13 only, in order to achieve local color nuances in the veneer 9. In other words, in this case, some of the volume elements 13 are filled with the second ceramic paste 26a whereas the remaining volume elements 13 are filled with the first ceramic paste 26. Alternatively, one or more entire layers can be filled with the second paste. This is illustrated in FIG. 14 that shows a crowned tooth 1′, or more precisely, a crowned incisor. The veneer 9 of this crowned tooth 1′ comprises inner layers the volume elements 13 of which are filled with the first ceramic paste 26 only, and outer layers (incisal layers) the volume elements 13 of which are filled with the second ceramic paste 26a only. The second ceramic paste 26a optically mimics the dental enamel 2 whereas the first ceramic paste 26 optically mimics the dentin 3 in colour.

The first print head and the second print head are arranged adjacent to each other in the system 20 in such a way that the manipulator 21 can position the preform 6 either under the output opening of the first or the second print head. The control software is then set up to control the manipulator 21 in such a way that it aligns the preform 6 according to 105 either underneath the dispensing opening of the first or the second print head. This is done depending on a value of a parameter that is assigned to each volume element 13 or at least each layer 12. As the parameter needs to distinguish between two pastes only, the type of the parameter can be binary. For example, a value of 0 for the parameter associated to a volume element 13 indicates that this volume element 13 is to be filled with the first ceramic paste 26 and a value of 1 for the parameter indicates that the volume element 13 is to be filled with the second ceramic paste 26a. Similar, a value of 0 for the parameter associated to a layer 12 indicates that all volume elements 13 of this layer 12 are to be filled with the first ceramic paste 26 and a value of 1 for the parameter indicates that all volume elements 13 of this layer 12 are to be filled with the second ceramic paste 26a. The association between the parameter and the individual volume elements or layers can be part of a file or a database that is accessed by the control software 57. The association can particularly be a part of the layer structure.

For the skilled person it is obvious that the above concept can be extended to three, four or more ceramic pastes with different optical and/or mechanical properties. These ceramic pastes will then each be dispensed by a separate print head, to which the manipulator aligns the preform in step 105 as required or depending on the value of the parameter.

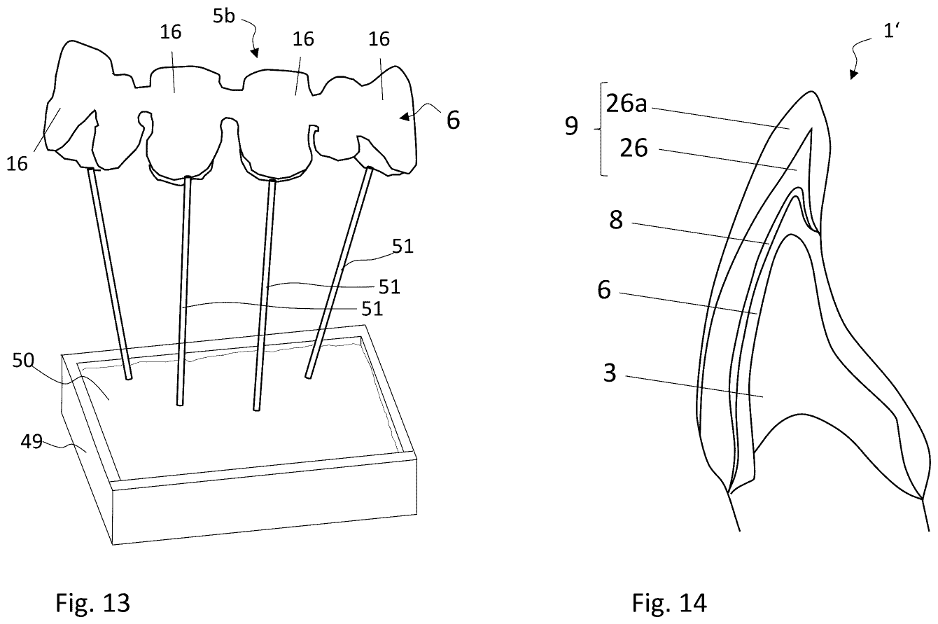

Now turning to FIG. 13, the way the preform 6 is held is explained. Here, the preform 6 is again a pontic 5b with four main bodies 16. In order to ensure that the preform 6 is accessible from as many directions as possible it is held at a distal end of at least one stick 51. As illustrated in FIG. 13, four sticks 51, one for each main body 16 are used. The sticks 51 are made of metal and have a length of approximately 40 mm and a diameter of 1 mm. The respective proximate end of the sticks opposite the distal end is cemented in an embedment compound 50 that is an initially plastically deformable, but curing mass provided in an embedment container 49. After curing the embedment compound 50 holds the sticks 51 firmly.

The container 49 comprises a bottom and a frame enclosing an interior filled with the embedment compound 50. In the embodiment shown in FIG. 13, the frame has a rectangular shape. However, other shapes are possible as well, i.e. triangular, round, oval, or trapezoidal for example. The underside of the container's bottom has a flat surface so that the container 49 can be placed stable on a flat surface.

The preform 6, the sticks 51 and the embedment container 49 with embedment compound 50 form a simply manageable unit that can easily be attached to the manipulator's carrier 30, see FIG. 8b. Thus, the preform is not held directly on the manipulator 21 or carrier 40, but indirectly via the sticks 51 and the embedment container 49. For this purpose the container 49 is positioned in the clamping or gripping mechanism 39 by opening the clamping jaws 39a, 39b against the spring force and placing the container 49 between the clamping jaws 39a, 39b that then firmly hold it.

The method according to the present invention is a true 3D printing process because the preform 6 can be moved, or moves simultaneously, in all spatial directions x, y, z for the application of the portions 19 of ceramic paste 26 in the volume elements 13 of a layer 12. More precisely, the preform 6, by means of the manipulator 21, is moved forward, backwards, to the left, to the right, it can be lowered and lifted, relative to the print head 22. In addition, the preform 6 can also be rotated around multiple axis. Only in this way it is possible to print on the preform 6 layer by layer which is as a complex shaped body, which is not feasible with conventional methods. This makes the present invention significantly different from all known ceramic printing method.

It should be noted that the foregoing description is given by way of example only for the purpose of illustration and in no way limits the scope of protection of the invention. Features of the invention which are indicated as “may”, “exemplary”, “preferred”, “optional”, “ideal”, “advantageous”, “optionally”, “suitable” or the like are to be regarded as purely optional and also do not limit the scope of protection, which is defined exclusively by the claims. Insofar as elements, components, process steps, values or information are mentioned in the above description which have known, obvious or foreseeable equivalents, these equivalents are included in the invention. Likewise, the invention includes any changes, variations or modifications of embodiments which involve the replacement, addition, alteration or omission of elements, components, process steps, values or information, as long as the basic idea of the invention is retained, regardless of whether the change, variation or modification leads to an improvement or deterioration of an embodiment.

Although the above description of the invention mentions a large number of physical, non-physical or process-related features in relation to one or more specific embodiment example(s), these features can also be used in isolation from the specific embodiment example, at least insofar as they do not require the mandatory presence of further features. Conversely, these features mentioned in relation to one or more specific embodiment example(s) can be combined with each other and with other disclosed or undisclosed features of disclosed or undisclosed embodiment examples as desired, at least insofar as the features are not mutually exclusive or do not lead to technical incompatibilities.

### REFERENCE LIST

1 Natural tooth
1a Tooth head
1b Tooth root
1c Tooth trunk
1′ Crowned tooth
2 Dental enamel
3 Dentin
4 Nerve
5 Crown, dental prosthesis with ceramic base
5a Crown, dental prosthesis with metallic base
5b Pontic, dental prosthesis
6 Preform, hard-ceramic base
6a Metallic base
7 Bonding agent
8 Color layer/Opaquer
9 Veneer, dental ceramic
10 Gloss Layer
11 Layer Structure
12 Layer
12a First layer
12b Second layer
13 Volume element, Voxel
14 Application point
15 free
16 Main body
17 Bridge member
18 Inner edge
19 Portion of ceramic paste
20 System
21 Manipulator
22 Print head
23 Scanner
24 Blower
25 Container for paste/ceramic slurry
26 Paste/ceramic slurry
26a Second paste, second ceramic slurry
27 Nozzle
27a Dispensing opening
27a Ejection channel
28 Camera
29 Dehumidifyer
30 Heater
31 Base plate
32 Mounting rod
33 Supporting arm
34 Mounting rod
35 Mounting rod
36 Manipulator foot
37 Multi-section arm
37a Lower Arm
37b Middle Arm
37c Upper Arm
38 Pivot bearing
38a . . . 38g first to seventh pivot bearing
39 Clamp
39a, 39b First and second clamp portion
40 Carrier
41 Heated air
42 Control unit
43 Control line
44 Cleaning apparatus
45 Motor
46 Shaft
47 Propeller
48 Wing
49 Embedment container
50 Embedment compound
51 Metal stick
52 Valve element
53 Valve opening
54 Actuator
55 Computing unit
56 Processing software
57 Control software
58 Inner geometry of layer structure
59 Outer Geometry of layer structure
60 Outer Geometry of preform
61 Incircle
62 Ground surface
63 Normal vector
64 Shaft axis
65 Evaluation unit
66 Chamber
101-112 Method steps
R Ejection direction
A-G Directions of movement
B1 . . . B4 Pivotal axis
α, β angle

1. A Method for the automated, software-based manufacturing of a veneer of a
fixed dental prosthesis by coating a dental preform with at least one compound, which serves to reproduce the dentin of a natural tooth, wherein the compound is a ceramic paste which is formed from a powder of a feldspar ceramic finely dispersed in a solvent, the method comprising the steps of:
a) generating, from image data, a virtual, three-dimensional layer structure which corresponds in shape and size substantially to a spatial volume difference between a first spatial volume and a second spatial volume that is smaller than the first spatial volume, the first spatial volume being the spatial volume of that part of the natural tooth or group of teeth to be replaced by the dental prosthesis, and the second spatial volume being the spatial volume of the preform, the spatial volume difference being intended to be filled with the ceramic paste, wherein the layer structure is divided, in the radial direction, into layers and the layers are divided, in the lateral direction, into volume elements, each volume element being intended to be filled with a single portion of the ceramic paste,
b) providing the preform on a carrier that is held by a multi-axis manipulator configured to move and align the preform in space,
c) providing the paste for a print head which is configured to vertically eject individual portions of the paste from a dispensing opening in an ejection direction,
d) coating the preform by filling at least some of the volume elements of the layer structure successively with a single portion of the ceramic paste, comprising, for each volume element, the substeps of:
i. the manipulator aligning the preform in such a way that a ground surface of the volume element to be filled, at an application point on said ground surface, is oriented substantially perpendicular to the ejection direction with the application point lying on the ejection direction,
ii. the print head applying the respective portion of the paste by jerking a valve element in the print head so that the portion is formed and projected in a coherent manner in the direction of the application point and fills the volume element in a punctiform manner,
e) transporting the coated preform into an oven and
f) Firing the coated preform in the oven.

2. The method according to claim 1, wherein an STL-file is used to determine the spatial volume difference, calculate the amount of locally necessary layers and to generate the layer structure.

3. The method according to claim 1, wherein the size of the volume elements is such that the portions filled in two neighbouring volume elements within the same layer can flow into each other.

4. The method according to claim 1, wherein the dental prosthesis is a pontic intended to replace the visible parts of two or more natural teeth, and the preform consists of a number of main bodies corresponding to the number of teeth to be replaced, which are integrally connected to one another by a narrow connecting element, that is coated first with the ceramic paste.

5. The method according to claim 1, wherein filling the volume elements of the layer structure with a portion of the ceramic paste is carried out layer by layer.

6. The method according to claim 1, wherein the distance between the dispensing opening of the print head and the application point of the respective volume element should not exceed a maximum value of 8mm.

7. The method according to claim 1, wherein sub-step d)ii. is repeatedly executed with a cycle frequency between 0.5 Hz and 5 Hz.

8. The method according to claim 1, wherein the step of aligning the preform is performed continuously by the manipulator, so that moving the preform is not stopped for the execution of sub-step d)ii.

9. The method according to claim 1, further comprising the step of providing a blower that blows heated and/or dehumidified air onto the preform.

10. The method according to claim 1, further comprising the step of providing a cleaning device with at least one wing that can be pivoted relative to the dispensing opening, and operating the cleaning device so that the at least one wing sweeps over the dispensing opening thereby shearing off accumulated ceramic paste from the dispensing opening.

11. The method according to claim 1, further comprising the step of providing an optical sensor that is arranged in such a way that it detects the dispensing opening, the optical sensor transmitting sensor data associated with the dispensing opening to an evaluation unit, the evaluation unit evaluating the sensor data to determine whether ceramic paste has accumulated at the dispensing opening and, if this is the case, triggering the control unit to transmit a respective control command.

12. The method according to claim 1, wherein at least one additional layer is provided in the layer structure in that the layer structure is larger than the spatial volume difference by this additional layer or layers in order to take the firing-related shrinkage of the ceramic paste in step f) into account.

13. The method according to claim 1, wherein step d) of the method is carried out firstly for only some of the volume elements of the layer structure, and steps d), e) and f) are repeated for a second part of the volume elements or for all remaining volume elements.

14. The method according to claim 1, wherein the manipulator presents the partly or fully coated preform, before or after firing, in several previously defined positions to a scanner having at least two cameras, and the scanner optically captures the coated preform in different perspectives while a striped light pattern consisting of alternating light and dark stripes in parallel is projected onto the coated preform in each of the presented positions, and an evaluation unit determines reference points on the surface of the coated preform and compares them with the generated layer structure in order to detect deviations in the size and/or geometry of the preform compared to the layer structure.

15. The method according to claim 1, wherein the compound comprises ammonium polyacrylate, ethoxylated castor oil and traces of urea.

16. A compound for manufacturing of a veneer of a fixed dental prosthesis, whereas the compound is a ceramic paste formed from a powder of a feldspar ceramic finely dispersed in a solvent and comprising amounts of ammonium polyacrylate, ethoxylated castor oil and traces of urea.

17. The compound according to claim 16, wherein the ammonium polyacrylate has an amount of about 2.2 percent by weight relative to the solids content.

18. The compound according to claim 16, wherein the ethoxylated castor oil has an amount of approx. 0.4 percent by weight.

19. The compound according to claim 16, wherein the feldspar ceramic powder has an amount of around 65.4 percent by weight.

20. The compound according to claim 16, wherein the solvent has an amount of about 32 percent by weight relative to the total weight of the ceramic paste.

21. The compound according to claim 16, wherein the solvent is destilled water.

22. The compound according to claim 16, wherein the ceramic particles of the powder have a maximum particle diameter of 20 μm.

23. A System is proposed for the automated, software-based manufacturing of a veneer of a fixed dental prosthesis by coating a dental preform with at least one compound, which serves to reproduce the dentin of a natural tooth, the compound being a ceramic paste formed from a powder of a feldspar ceramic finely dispersed in a solvent, the system comprising:
a computing unit and processing software executable thereon, the software being configured to generate from image data a virtual, three-dimensional layer structure which corresponds in shape and size substantially to a spatial volume difference between a first spatial volume and a second spatial volume that is smaller than the first spatial volume, the first spatial volume being the spatial volume of that part of the natural tooth or group of teeth to be replaced by the dental prosthesis, and the second spatial volume being the spatial volume of the preform, the spatial volume difference being intended to be filled with the ceramic paste, wherein the layer structure is divided, in the radial direction, into layers and the layers are divided, in the lateral direction, into volume elements, each volume element being intended to be filled with a portion of the ceramic paste,
a carrier designed to hold the preform,
a multi-axis manipulator that can be moved in space and is designed to hold the carrier,
at least one print head comprising
a chamber for storing the paste to be ejected, the chamber having a valve opening,
a valve formed by a valve element and the valve opening of the chamber, wherein the valve opening can be sealed by the valve element on the inside of the chamber,
an actuator which is operatively connected to the valve element and is set up to move the valve element jerkily so that individual portions of the ceramic paste are formed and ejected from a dispensing opening in an ejection direction, the dispensing opening being the valve opening or an opening spaced apart from it,
whereby the print head is arranged in such a way that the ejection direction is vertical,
a control unit and a control software executable thereon, the control unit being communicatively connected to the manipulator and the print head and the control software being configured to control the manipulator and the print head depending on the layer structure in such a way that at least some of the volume elements of the layer structure are successively filled with a single portion of the ceramic paste, and in that for each volume element:
the manipulator aligns the preform in such a way that a ground surface of the volume element to be filled, at an application point on said ground surface, is oriented substantially perpendicular to the ejection direction with the application point lying on the ejection direction,
the print head applies the respective portion of the paste by jerking a valve element in the print head so that the portion is formed and projected in a coherent manner in the direction of the application point and fills the volume element in a punctiform manner,
an oven for firing the coated preform.

24. The system according to claim 23, wherein the actuator is an electromagnetic or piezoelectric drive.

25. The system according to claim 23, wherein the valve element is rod-like in the manner of a piston or plunger and comprises a head capable to sealingly close the valve opening.

26. The system according to claim 23, wherein dispensing opening has a diameter of between 200 μm and 500 μm.

27. The system according to claim 23, wherein the manipulator is configured to move the preform along all three spatial axes x, y, z and to rotate it about at least two axes of rotation.

28. The system according to claim 23, further comprising a scanner, which has at least two cameras directed at the preform held at the manipulator in order to capture stereoscopic images of the preform, the scanner being connected to the control unit in order to transmit images of the captured preform to the control software, and the control software is configured to determine the exact geographic position of the preform relative to a fixed point.

29. The system according to claim 23, further comprising a blower with a heater and/or a dehumidifier, the blower being positioned and directed in such a way that it blows heated and/or dehumidified air onto the preform.

30. The system according to claim 23, further comprising a cleaning device with at least one wing that can be pivoted relative to the dispensing opening in that when operating the at least one wing sweeps over the dispensing opening thereby shearing off accumulated ceramic paste from the dispensing opening.

31. The system according to claim 23, wherein the cleaning device comprise a servo motor and a shaft, the shaft being pivotably or rotatably driven around its axis by the servo motor when actuated, the at least one wing projecting from the free end of the shaft that is associated with the dispensing opening.

32. The system according to claim 23, further comprising an optical sensor that is arranged in such a way that it records sensor associated with the dispensing opening, the sensor being connected to an evaluation unit for transmitting the sensor data, the evaluation unit being configured to evaluate the sensor data to determine whether ceramic paste has accumulated at the dispensing opening and, if this is the case, to trigger a respective control command.

33. The system according to claim 23, wherein the preform is held at a first end of at least one stick, the other end of which is embedded in an embedment compound provided in an embedment container, whereas the embedment container is detachably attached to the manipulator's carrier.

34. The system according to claim 33, wherein the carrier is configured to hold the container by a latching or clamping mechanism.
